(19) United States
(12) Patent Application Publication
(10) Pub. No.: US 2023/0407222 A1
(43) Pub. Date: Dec. 21, 2023

(54) TEMPERATURE CALIBRATION METHODS AND DEVICES FOR USE IN AUTOMATED BIOREACTORS

(71) Applicants: OCTANE BIOTECH INC., Kingston, Ontario (CA); LONZA WALKERSVILLE, INC., Walkersville, MD (US)

(72) Inventors: Paul GARDIN, Kingston, Ontario (CA); Raelyn DANIELS, Kingston, Ontario (CA); Talib ALI, Kingston, Ontario (CA); Guy ORAM, Kingston, Ontario (CA); Matthew HEWITT, Walkersville, MD (US); Eytan ABRAHAM, Walkersville, MD (US)

(21) Appl. No.: 18/247,406

(22) PCT Filed: Oct. 7, 2021
(86) PCT No.: PCT/US2021/071773

§ 371 Date: Mar. 30, 2023

Related U.S. Application Data
(60) Provisional application No. 63/089,840, filed on Oct. 9, 2020.

(51) Int. Cl. C12M 3/00; C12M 1/34; C12M 1/36

(52) U.S. Cl. CPC C12M 23/42; C12M 41/12; C12M 41/48

(57) ABSTRACT

The present disclosure provides devices and associated methods for temperature monitoring and control in automated biological material engineering systems, including cell engineering systems. The devices and methods utilize measurement of internal temperatures in an automated system to map temperatures during the various processes carried out in the systems.

### CROSS-REFERENCE TO RELATED APPLICATION(S)

The present application claims priority to U.S. Provisional Application 63/089,840, entitled “Temperature Calibration Methods and Devices for Use in Automated Bioreactors” and filed Oct. 9, 2020, the entire content of which is incorporated by reference herein.

### FIELD OF THE INVENTION

The present disclosure provides devices and associated methods for temperature monitoring and control in automated biological material engineering systems, including cell engineering systems. The devices and methods utilize measurement of internal temperatures in an automated system to map temperatures during the various processes carried out in the systems.

### BACKGROUND OF THE INVENTION

As anticipation builds about accelerated clinical adoption of advanced cell and biomaterial therapies, more attention is turning to the underlying manufacturing strategies that will allow these therapies to benefit patients worldwide. While cell therapies hold great promise clinically, high manufacturing costs relative to reimbursement present a formidable roadblock to commercialization. Thus, the need for cost effectiveness, process efficiency and product consistency is driving efforts for automation in numerous cell therapy fields.

Automation of various processes is involved in producing cell populations for therapy. This includes integration of cell activation, transduction and expansion into a commercial manufacturing platform, for the translation of these important therapies to the broad patient population.

During the various processes of biological material manufacturing, including cell production, it is necessary to monitor temperature changes and gradients in such automated systems to ensure the biological processes are being carried out at the correct temperatures and that materials for use are being stored at the proper temperatures. The present invention fulfills these needs.

### SUMMARY

One aspect of the present disclosure relates to a calibration cassette for use in an automated biological material engineering system. The calibration cassette comprises a low temperature chamber, a high temperature chamber, one or more fluidics pathways, and electrical connection elements. The low temperature chamber includes a media storage vessel and a first array of sealed temperature probes in the media storage vessel. The high temperature chamber is separated from the low temperature chamber by a thermal barrier, the high temperature chamber including a cell culture chamber and a second array of sealed temperature probes in the cell culture chamber. The one or more fluidics pathways is connected to the cell culture chamber and the media storage vessel, and including a third array of sealed temperature probes in the one or more fluidics pathways. The electrical connection elements are electrically connected to each of the first, second, and third arrays of sealed temperature probes.

One aspect of the present disclosure relates to a production cassette for use in an automated cell engineering system. The production cassette includes a low temperature chamber including a cell culture media storage vessel and a first array of sealed temperature probes in the cell culture media storage vessel; a high temperature chamber for carrying out activation, transduction and/or expansion of a cell culture, the high temperature chamber separated from the low temperature chamber by a thermal barrier, the high temperature chamber including a cell culture chamber and a second array of sealed temperature probes in the cell culture chamber; one or more fluidics pathways connected to the cell culture chamber and the cell culture media storage vessel, and including a third array of sealed temperature probes in the one or more fluidics pathways; and electric connection elements that are electrically connected to each of the first, second, and third arrays of sealed temperature probes, wherein the one or more fluidics pathways provide recirculation, removal of waste, and homogenous gas exchange and distribution of nutrients to the cell culture chamber.

One aspect of the present disclosure relates to a method of temperature monitoring in an automated biological material engineering system. The method comprises receiving, by a control circuit, a set of internal temperature measurements during a time period when a first cassette is housed in the automated biological material engineering system, wherein the set of internal temperature measurements indicate temperature within the first cassette, and are generated during the time period by an array of temperature probes disposed within the first cassette; receiving, by the control circuit, an ambient temperature measurement when the first cassette is housed in the automated biological material engineering system, wherein the ambient temperature measurement indicates temperature outside the first cassette, and is generated during the time period by a system temperature probe of the automated biological material engineering system that is disposed outside the first cassette; and determining, by the control circuit, a set of temperature offset values that indicate respective differences between the set of internal temperature measurements and the ambient temperature measurement.

One aspect of the present disclosure relates to a method of temperature control performed in automated biological material engineering system. The method comprises receiving, by a control circuit, a set of internal temperature measurements during a first time period when a first cassette is housed in the automated biological material engineering system, wherein the set of internal temperature measurements indicate temperature within the first cassette, and are generated during the first time period by an array of temperature probes disposed within the first cassette; receiving, by the control circuit, a first ambient temperature measurement when the first cassette is housed in the automated biological material engineering system, wherein the first ambient temperature measurement indicates temperature outside the first cassette, and is generated during the first time period by a system temperature sensor of the automated biological material engineering system that is disposed outside the first cassette; determining, by the control circuit, a set of temperature offset values that indicate respective differences between the set of internal temperature measurements and the first ambient temperature measurement; determining, by the control circuit, a target internal temperature value for a location in a second cassette; and controlling, by the control circuit during a second time period when the second cassette is housed in the automated biological material engineering system, a heating device or cooling device of the automated biological material engineering system based on the target internal temperature value, the set of temperature offset values, and one or more additional ambient temperature measurement generated by the system temperature sensor during the second time period, wherein the system temperature sensor is disposed outside the second cassette.

### BRIEF DESCRIPTION OF THE FIGURES

FIG. 1 shows various steps that can be performed with a cassette of an automated biomaterial engineering system, as described in embodiments hereof.

FIG. 2A shows an exemplary cassette in accordance with embodiments hereof.

FIGS. 2B-2D show an exemplary cassette for use in temperature measurement and/or calibration, as described herein.

FIG. 2E shows a sealed temperature probe in accordance with embodiments hereof.

FIGS. 3A and 3B show images of an automated biomaterial engineering system in accordance with embodiments hereof.

FIGS. 3C and 3D show images of an automated biological material engineering system and a cassette, in accordance with embodiments hereof.

FIG. 3E show an image of a cassette in accordance with an embodiment hereof.

FIG. 3F shows an image of an automated biological material engineering system, a cassette, and a computing device, in accordance with an embodiment hereof.

FIG. 3G shows an image of an automated biological material engineering system that is able to receive a first cassette and a second cassette, in accordance with an embodiment hereof.

FIG. 4 shows a lab space containing exemplary biomaterial engineering systems as described in embodiments hereof.

FIG. 5 shows a flowpath for an automated biomaterial engineering system as described in embodiments hereof.

FIG. 6 depicts a flow diagram of an example method for determining temperature offset, in accordance with an embodiment hereof.

FIGS. 7A and 7B show a cassette for use in temperature measurement and/or calibration, as described herein.

FIGS. 8A and 8B show temperature offset values, in accordance with an embodiment herein.

FIG. 9 depicts a cassette that may be disposed within an automated biological material engineering system, in accordance with an embodiment herein.

FIGS. 10A and 10B depict a control temperature and a media temperature being affected by operation of a heating device, in accordance with an embodiment herein.

### DETAILED DESCRIPTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

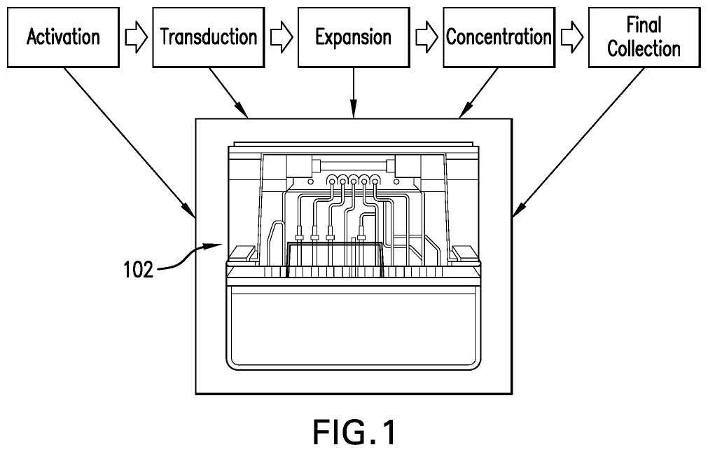

The methods, devices and systems provided herein are described in reference to their application with cassettes for use in automated biological material engineering systems. FIG. 1 shows an exemplary cassette 102, in which various processes can be carried out in an enclosed, automated system that allows for production of various samples and populations, and in particular “biological materials” that include proteins, peptides, antibodies, antibody fragments, as well as cells. Such processes can include activating, transducing, expanding, concentrating, washing, and collecting/harvesting steps of proteins and/or cells.

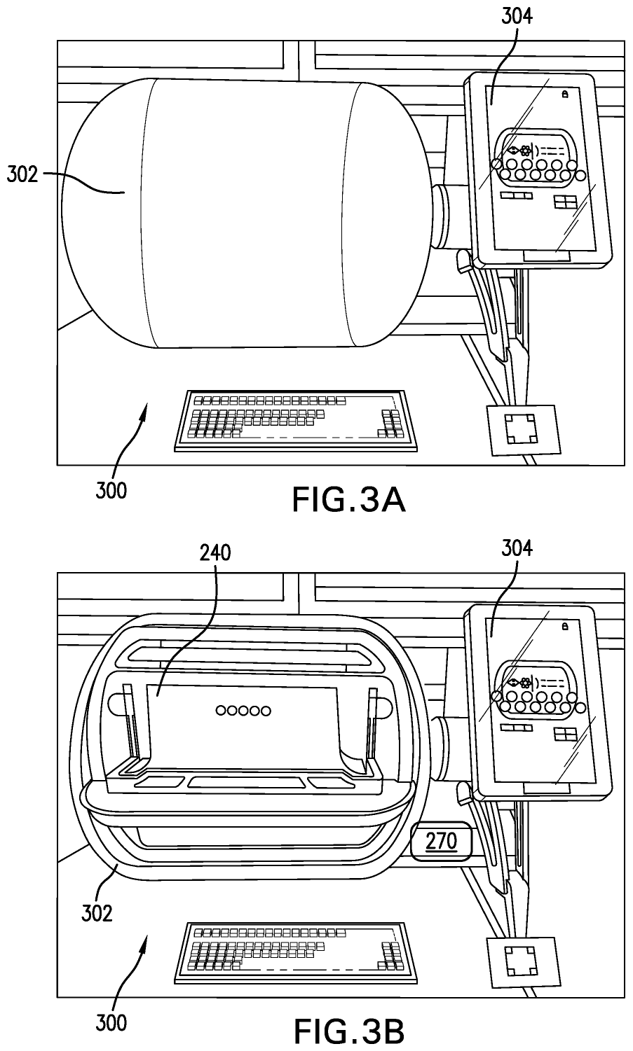

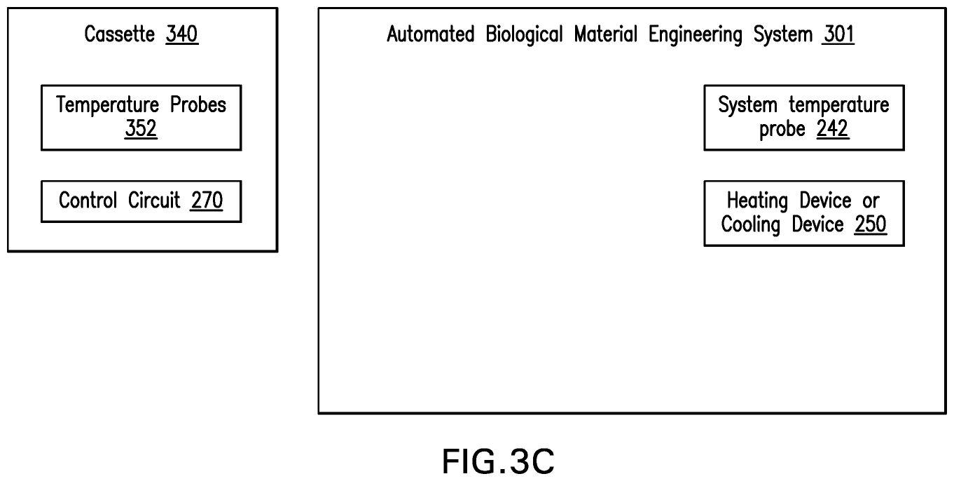

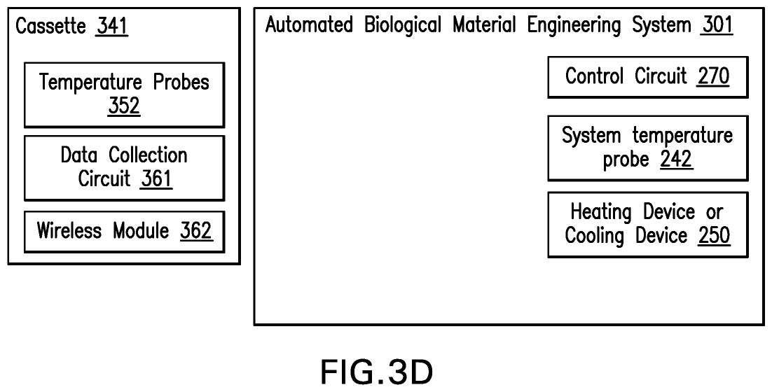

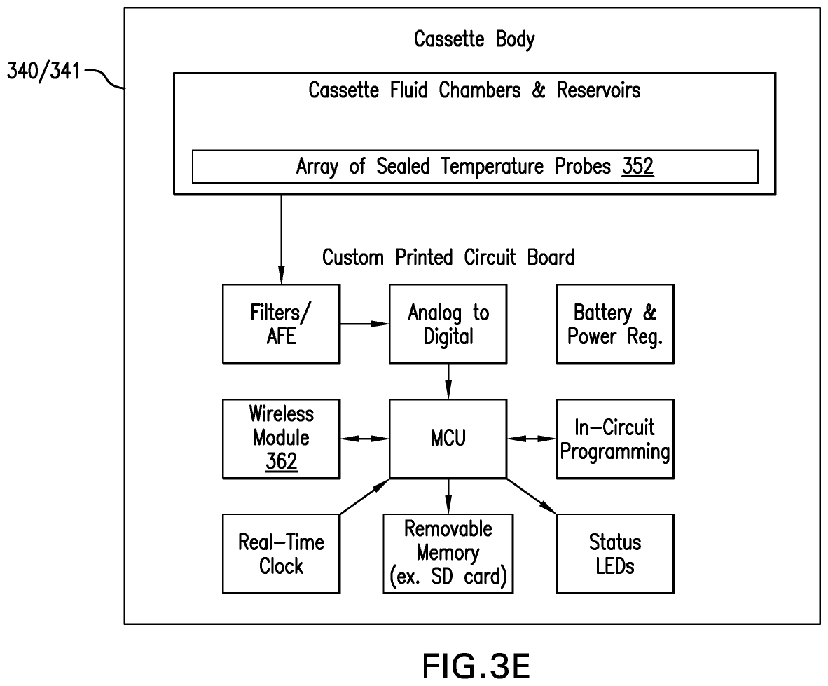

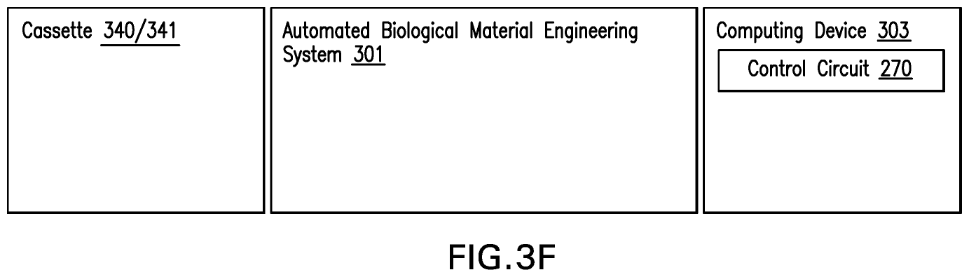

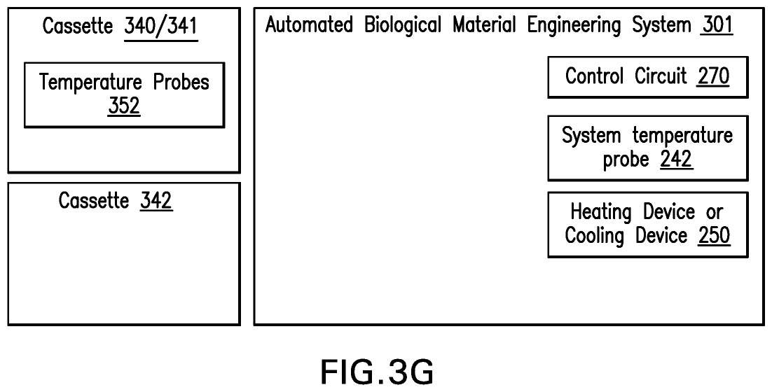

As described herein, the cassettes and methods are suitably utilized and carried out in a fully enclosed automated biological material engineering system 300 (see FIGS. 3A, 3B), including automated cell engineering systems, suitably having instructions thereon for performing steps such as, activating, transducing, expanding, concentrating, and harvesting. Cell engineering systems for automated production of, for example genetically modified immune cells, including CAR T cells, are described in U.S. patent application Ser. No. 16/119,618, filed Aug. 31, 2018 (the disclosure of which is incorporated by reference herein in its entirety), and is also called automated cell engineering system, COCOON®, or COCOON™ system herein.

For example, a user can provide an automated cell engineering system pre-filled with a cell culture and reagents (e.g., an activation reagent, a vector, cell culture media, nutrients, selection reagent, and the like) and parameters for the cell production (e.g., starting number of cells, type of media, type of activation reagent, type of vector, number of cells or doses to be produced, and the like). The automated cell engineering system is able to carry out the various automated methods, including methods of producing genetically modified immune cell cultures, including CAR T cells, without further input from the user. In some embodiments, the fully enclosed automated cell engineering system minimizes contamination of the cell cultures by reducing exposure of the cell culture to non-sterile environments. In additional embodiments, the fully enclosed automated cell engineering system minimizes contamination of the cell cultures by reducing user handling of the cells.

The automated engineering systems can also be used to prepare other biological materials, including various proteins, peptides, antibodies, antibody fragments, etc.

As described herein, the automated biomaterial engineering systems 300 suitably include a cassette 102. As used herein a “cassette” refers to a largely self-contained, removable and replaceable element of an automated biomaterial engineering system that includes one or more chambers for carrying out the various elements of the methods described herein, and suitably also includes one or more of a cell media, an activation reagent, a wash media, etc.

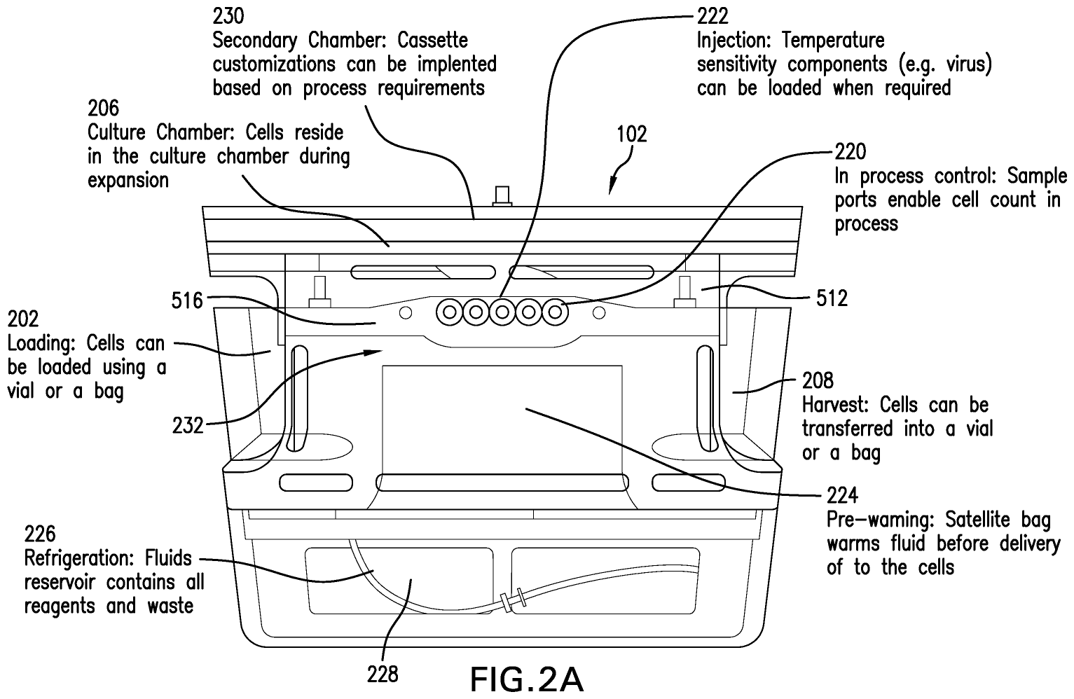

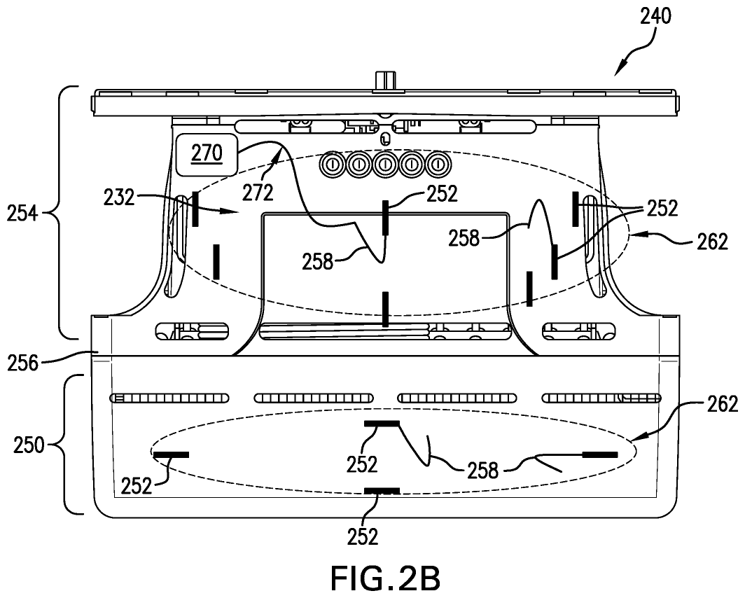

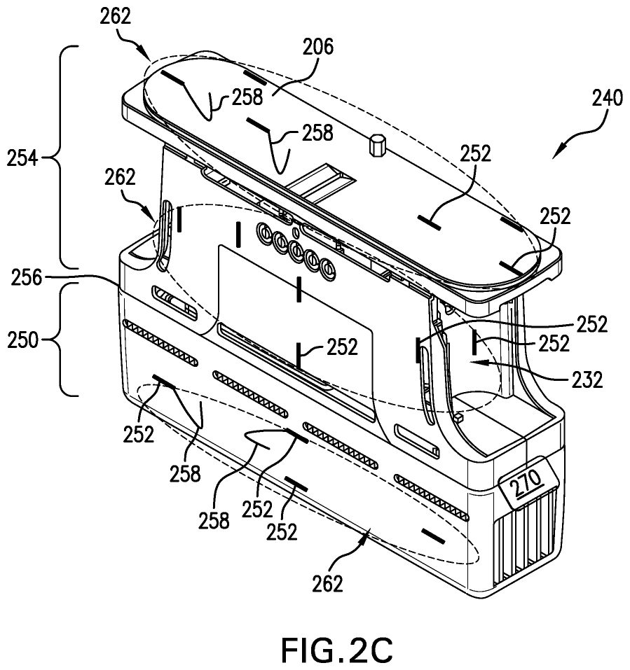

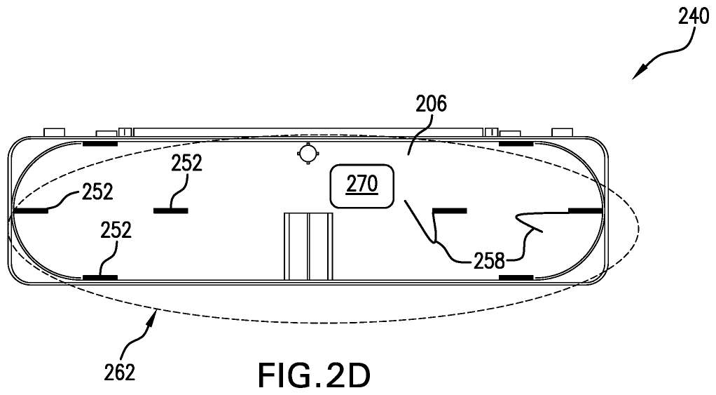

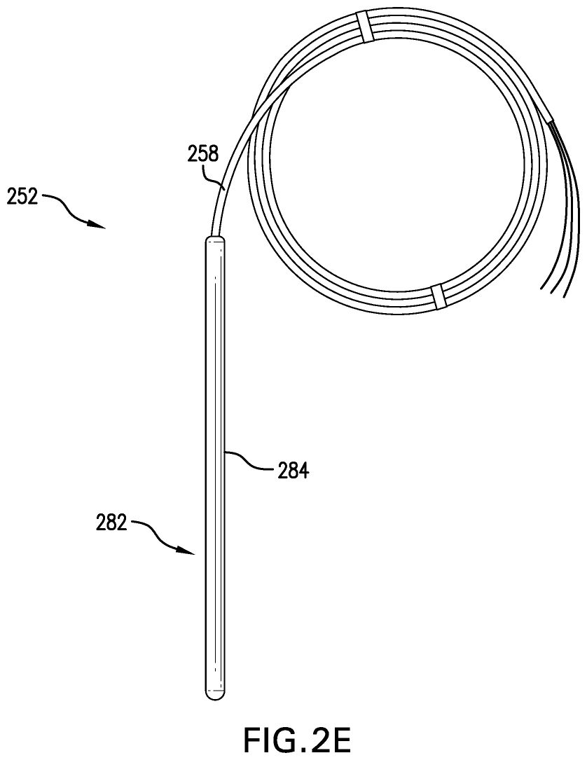

FIG. 2A shows an exemplary cassette 102 for use in an automated biomaterial engineering system, including an automated cell engineering system. In embodiments, cassette 102 includes a cellular sample input 202. Cellular sample input 202 is shown in FIG. 2A as a vial or chamber in which a cellular sample can be placed prior to introduction or loading into cassette 102. In other embodiments, cellular sample input 202 can simply be a sterile-locking tubing (for example a luer lock tubing connection or the like) to which a syringe or a cell-containing bag, such as a blood bag, can be connected.

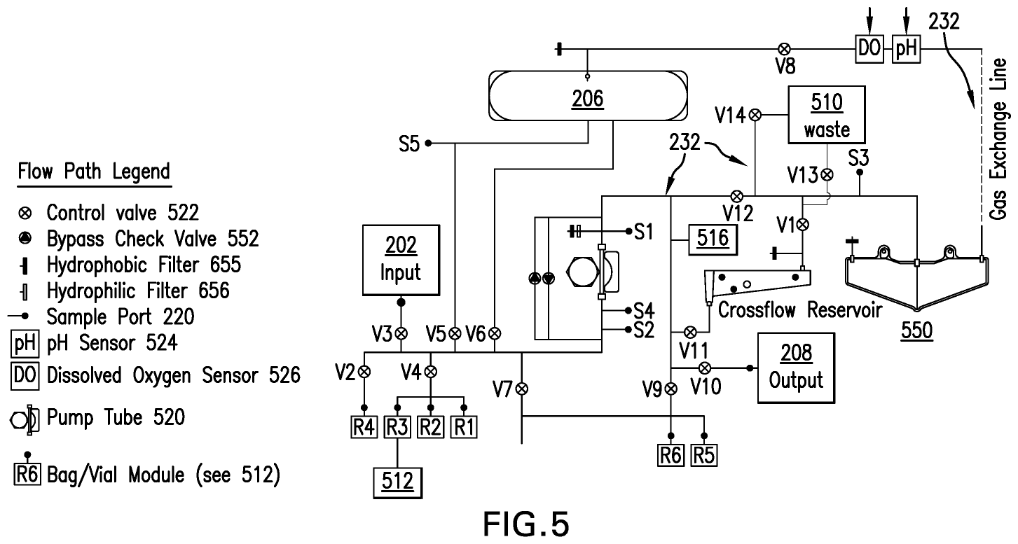

Cassette 102 further includes a cell culture chamber 206. Examples of the characteristics and uses of cell culture chamber 206 are described herein. Cassette 102 also includes a pumping system 520 (see FIG. 5 for exemplary location in the flowpath) fluidly connected to cell culture chamber 206.

As used herein, “fluidly connected” means that one or more components of a system, such as components of cassette 102, are connected via a suitable element that allows for fluids (including gasses and liquids) to pass between the components without leaking or losing volume. Exemplary fluid connections include various tubing, channels and connections known in the art, such as silicone or rubber tubing, luer lock connections, etc. It should be understood that components that are fluidly connected can also include additional elements between each of the components, while still maintaining a fluid connection. That is, fluidly connected components can include additional elements, such that a fluid passing between the components can also pass through these additional elements, but is not required to do so.

Pumping system 520 is suitably a peristaltic pump system, though other pumping systems can also be utilized. A peristaltic pump refers to a type of positive displacement pump for pumping a fluid. The fluid is suitably contained within a flexible tube fitted inside a pump casing—often circular. A rotor with a number of “rollers”, “shoes”, “wipers”, or “lobes” attached to the external circumference of the rotor compresses the flexible tube. As the rotor turns, the part of the tube under compression is pinched closed (or “occludes”) thus forcing the fluid to be pumped to move through the tube. Additionally, as the tube opens after the passing of the cam (“restitution” or “resilience”) fluid flow is induced to the pump. This process is called peristalsis and is used to move fluid through the flexible tube. Typically, there are two or more rollers, or wipers, occluding the tube, trapping between them a body of fluid. The body of fluid is then transported toward the pump outlet.

In embodiments, cassette 102 further includes one or more fluidics pathways suitably connected to the cell culture chamber (see 232 inside cassette 102 in FIG. 2A). Also included in cassette 102 is a cellular sample output 208 fluidly connected to cell culture chamber. As described herein, cellular sample output 208 can be utilized to harvest the cells following the various automated procedures for either further processing, storage, or potential use in a patient, or for further processing to isolate a desired protein or peptide that the cells are producing. Cellular sample output 208 can also be a sample port 220, as described herein, that allows a cellular sample to be removed from the cassette, for example for transduction such as electroporation, and then returned to the cassette for further automated processing. Examples of fluidics pathways 232 include various tubing, channels, capillaries, microfluidics elements, etc., that provide nutrients, solutions, etc., to the elements of the cassette, as described herein.

In exemplary embodiments, provided herein is a cassette for temperature measurement and/or calibration 240 for use in an automated biological material engineering system. Cassette 240 can be used as a calibration cassette or a production cassette. A “calibration” cassette refers to a cassette that is not utilized during the production of a biological material, and thus simply serves as a trial or dummy cassette for the measurement of temperature variations and gradients during a process. A “production” cassette refers to a cassette that can be utilized to carry out the production of a biological material, including cells. Both calibration cassettes and production cassettes are discussed herein with regard to cassette 240, as shown in the Figures.

As shown in FIG. 2B, cassette 240 suitably includes a low temperature chamber 250 including a media storage vessel 228 (see FIG. 2A) and a first array 262 of sealed temperature probes 252 in the media storage vessel 228. Sealed temperature probes 252 are represented in FIGS. 2B-2D as short, solid lines. As used herein an “array” 262 of sealed temperature probes 252 refers to arrangement of a plurality of sealed temperature probes 252 that allows the probes to measure the temperature at multiple different points within a structure for ultimate use to map or describe the temperature profile of a surface, structure, vessel, body, etc. “Plurality” includes 2 or more of an item, including sealed temperature probes, and suitably includes 3 or more, 4 or more, 5 or more, 6 or more, 7 or more, 8 or more, 9 or more, or more, 15 or more, 20 or more, 25 or more, 30 or more, etc., of an item, such as sealed temperature probes 252.

In exemplary embodiments, each of the first, second, and third arrays of sealed temperature probes each include at least 2 sealed temperature probes. That is, each of the arrays configured to provide temperature information about a particular section of the cassette 240, including for example, the high temperature chamber 254, the low temperature chamber 250, and the fluidics pathway 232. In suitable embodiments, each of the different arrays 262 of sealed temperature probes 252 include between 2-20 sealed temperature probes, more suitably between 2-15 sealed temperature probes, between 2-10, between 2-9, between 2-8, between 2-7, between 2-6, between 2-5, between 2-4, or 2-3 sealed temperature probes in each array. In embodiments, the total number of sealed temperature probes 252 in all of the arrays 262 is suitably 10-15 probes, more suitably 10, 11, 12, 13, 14 or 15 total probes.

As shown in FIG. 2B, cassette 240 also suitably includes a high temperature chamber 254 separated from the low temperature chamber 250 by a thermal barrier 256. Suitably, the high temperature chamber 254 includes a cell culture chamber 206 and a second array 262 of sealed temperature probes 252 in the cell culture chamber 206 (see FIG. 2C showing a side view of cassette 240; FIG. 2D shows a top view of cassette 240.

In embodiments, low temperature chamber 250, can include a refrigeration area 226 suitably for storage of a cell culture media. High temperature chamber 254, suitably for carrying out activation, transduction and/or expansion of a cell culture, for example in cell culture chamber 206. Suitably, the high temperature chamber is separated from the low temperature chamber by thermal barrier 256, which can be an insulating layer, section or chamber, that maintains the temperatures between high and low temperature regions distinct. As used herein “low temperature chamber” refers to a chamber, suitably maintained below room temperature, and more suitably from about 4° C. to about 8° C., for maintenance of cell media, etc., at a refrigerated temperature. The low temperature chamber can include a bag or other holder for media, including about 1 L, about 2 L, about 3 L, about 4 L, or about 5 L of fluid. Additional media bags or other fluid sources can be connected externally to the cassette, and connected to the cassette via an access port. The brackets shown in FIGS. 2B and 2C showing the location of low temperature chamber 250 and high temperature chamber 254 are representative and are not limiting to as to the full dimensions of either chamber, but are instead provided to represent a suitable location and reference dimensions for each chamber.

As used herein “high temperature chamber” refers to a chamber, suitably maintained above room temperature, and more suitably maintained at a temperature to allow for cell proliferation and growth, i.e., between about 35-40° C., and more suitably about 37° C. In embodiments, high temperature chamber suitably includes cell culture chamber 206 (also called proliferation chamber or cell proliferation chamber throughout).

In further embodiments of cassette 240, one or more fluidics pathways that are contained therein and connected to the cell culture chamber and the media storage vessel, also suitably include a third array 262 of sealed temperature probes 252 in the one or more fluidics pathways 232 (see FIGS. 2B and 2C).

As shown in FIGS. 2B-2D, sealed temperature probes 252 also suitably include electrical connection elements 258 that are electrically connected to each of the first, second, and third arrays of sealed temperature probes. Not all electrical connection elements 258 are shown in FIGS. 2B-2D to allow for easier viewing. It should be noted that suitably, each of the sealed temperature probes 252 include electrical connection elements 258 to allow for electrical connection of the sealed temperature probes to a source of electrical power and/or to allow communication of a measurement signal.

As used herein a “sealed temperature probe” refers to a device capable of measuring the temperature of a surface, solution or gas, and also includes a cover that encloses the probe and limits or suitably prohibits transfer of liquids and/or gasses across the cover. An exemplary sealed temperature probe 252 is shown in FIG. 2E, illustrating the temperature probe 282 (for measuring temperature) and cover (or seal) 284 (only cover can be seen as probe is within the cover), as well as electrical connection element 258. Suitable covers that can be utilized include various polymers, and suitably the cover provides a hermetic seal, i.e., an airtight seal, around the temperature probe. Exemplary temperature probes included resistance temperature detectors (RTD) hermetically sealed by a polymeric cover. Additional temperature probes that can be utilized include thermocouples and thermistors, that also suitably include a seal or cover to reduce or eliminate contamination of the probe with fluids or gasses, etc.

As described herein, in exemplary embodiments, media storage vessel 228 is a bag, and wherein the array 262 of sealed temperature probes 252 are attached to an internal surface of the bag. As described herein, suitably cell culture chamber 206 is flat and substantially non-flexible. It has been found that such a cell culture chamber allows for increased cell yield during a cellular production process or a biomaterial production process. In embodiments, the array 262 of sealed temperature 252 probes are attached to a bottom and/or a side of the cell culture chamber.

As described herein, suitably the fluidics pathways within the cassettes include various tubing and connectors, and in embodiments, also include a third array 262 of sealed temperature probes 252 are located within the tubing. See FIG. 2B in the middle section of the cassette 240, illustrating potential exemplary locations of sealed temperature probes 252 within the tubing. The location of temperature probes within the tubing can be designed so that temperature profiles and gradients can be measured in various section of the fluid pathway. Including for example, in tubing configured to deliver cell culture media to the cell culture chamber 206; in tubing configured to remove cells from cell culture chamber 206; in tubing configured to mix cell culture media so as to regulate gas content; in tubing configured to allow for sampling of the cell culture process or other biomaterial process; in tubing configured to provide input paths to the cell culture chamber 206; as well as other configurations. Methods for attaching sealed temperature probes 252 to one or more sections or elements of the cassettes described herein are known in the art and include for example, use of various adhesives, tapes, glues, heat sealing methods, soldering methods, connection with mechanical fasteners, as well as direct integration into the cassette elements such as during formation, molding or manufacturing.

Suitably, cassette 240 described herein further includes a control circuit 270 that is electrically connected to the electric connection elements 258 and thus to sealed temperature probes 252. FIG. 2B shows an exemplary location for control circuit 270, and its electrical connection 272 to one of the electric connection elements 258. It should be understood that other electric connection elements 258, and thus probes 252, can be connected to control circuit 270 in a similar manner, but are not shown in the figures to ease visualization. Control circuit 270 can also be placed in any desired location on cassette 240, including for example, along the side, bottom, or top of cassette 240 (see FIG. 2C for an exemplary side location; FIG. 2D for an exemplary top location), and can be located internally (i.e. inside of the housing) or externally to the cassette. The locations of control circuit 270 shown in the figures are for illustrative purposes only, and a person of ordinary skill in the art will readily understand that other locations and configurations can also be used. While the control circuit is suitably electrically wired to the temperature probes, some embodiments may involve a wireless connection (e.g., radio frequency, Bluetooth®, etc.) for communicating with the temperature probes. In such embodiments, another device may be electrically wired with the temperature probes via a wired connection, and may further have a wireless connection with the control circuit. That is, this device may be configured to wirelessly communicate with the control circuit. Such a device may, e.g., include a data collection circuit that has a wired connection to the temperature probes and configured to receive measurements or other data generated by the temperature probes, and may further include a wireless module (also referred to as a wireless communication circuit) that may relay such data to the control circuit via the wireless connection.

A “control circuit” as used herein refers to an electronic circuit that provides a functionality relating to temperature control and/or temperature measurement. The control circuit 270 may be configured to, e.g., receive and process temperature measurements made by the temperature sensors 252 or any other temperature sensor. In some embodiments, the control circuit may be configured to control a communication module (e.g., a wireless module) to communicate the temperature measurements to a computer system for temperature recording. In some cases, the control circuit may be configured to control or otherwise influence temperature modification (i.e. increasing or decreasing temperature), such as by controlling a heating device or a cooling device. In embodiments, control circuit 270 provides simply measurement and recording functions of the temperature of the cassette 240 to allow for mapping of the temperature, as described herein. In some embodiments, the control circuit 270 can also be utilized to record or log the temperature measurements over a set period of time for later use or extraction. In such embodiments, if the control circuit 270 is external to a computer, the control circuit does not need to be immediately connected to the computer, but instead can be later connected (either wirelessly or via direct connection) and the data transferred. In embodiments, control circuit 270 can be programmed to turn off or enter a low power state in between temperature measurements to reduce unintended heating of the cassette. In an embodiment, the control circuit 270 can include a processing circuit, such as one or more microprocessors, microprocessor cores, a programmable logic circuit (PLC), a field programmable gate array (FPGA) circuit, an application specific integrated circuit (ASIC), a microcontroller unit (MCU), and/or any other control circuit.

As discussed in more detail below, the control circuit 270 may be located in or otherwise associated with a cassette, an automated biological material engineering system, or some other device, such as a desktop computer or laptop in communication with the automated biological material engineering system. For instance, the control circuit 270 may be associated with the cassette 240, and thus is connected directly to cassette 240, contained within cassette 240, or made a part of cassette 240. In other embodiments, control circuit 270 can be associated with the automated biological material engineering system 300. For example, as shown in FIG. 3B, control circuit 270 can be connected to, contained within, or otherwise made a part of biological material engineering system 300. When the cassette 240 is inserted into the system 300, the cassette 240 may communicate with the control circuit via a wired electrical connection or a wireless connection. For instance, if the cassette has one or more electrical contacts or other electrical conductors (e.g., wires) which extend from a location inside the cassette to a location that is outside the cassette or a location on an outer surface of the cassette, the electrical conductors may provide the wired electrical connection to provide communication and/or to provide power to various components of the cassette. In such an example, the electrical connectors may include or be electrically connected to the electrical connection elements 258, which may be electrically connected to the temperature probes 252 in the cassette 240. If the electrical conductors are also electrically connected (directly or indirectly) to the control circuit 270, they may provide an electrical connection through which communication can occur between the control circuit 270 and the temperature probes 252. For example, this can occur by plugging in the electrical connection elements 258 into connectors which are electrically connected to the control circuit 270, and can take place as the cassette 240 is being inserted into the system 300 (i.e., as a plug and play connection) or done after the cassette 240 is inserted (i.e., as a further connection, perhaps used only if desired). As stated above, the communication (also referred to as a communicative connection or communicative coupling) between cassette 240 and control circuit 270 can also be carried out wirelessly, especially if the cassette 240 has no conductors which can provide a wired connection between a location inside the cassette 240 to a location outside the cassette 240.

Various filters or separation devices be utilized in the cassettes and methods described herein. For example, a magnetic separation process can be utilized to eliminate and separate undesired cells and debris from a cell population. In such embodiments, a magnetic bead or other structure, to which a biomolecule (e.g., antibody, antibody fragment, etc.) has been bound, can interact with a target cell. Various magnetic separation methods, including the use of filters, columns, flow tubes or channels with magnetic fields, etc., can then be used to separate the target cell population from undesired cells, debris, etc., that may be in a cellular sample. For example, a target cell population can flow through a tube or other structure and be exposed to a magnetic field, whereby the target cell population is retained or held-up by the magnetic field, allowing undesired cells and debris to pass through the tube. The magnetic field can then be turned off, allowing the target cell population to pass onto a further retention chamber or other area(s) of the cassette for further automated processing. Additional filtration includes traditional column filtration, or use of other filtration membranes and structures.

In embodiments where a magnetic separation process is utilized, the cassettes described herein can also further include a magnetic probe to measure and map the magnetic flux within and surrounding the cassette. This measurement and mapping provides information that can be utilized for calibration, verification and/or control of the magnetic field during the separation process.

In further embodiments, cassette 240 further includes a waste collection chamber 510. In additional embodiments, a satellite volume 550, which can be provide additional storage capabilities for the cassette, to increase the overall volume of the automated processes. An exemplary location of satellite volume 550 is shown in the flowpath of FIG. 5. The cassettes can also further include one or more fluidics pathways (generically 232), wherein the fluidics pathways provide recirculation, removal of waste and homogenous gas exchange and distribution of nutrients to various parts of the cassette, including the cell culture chamber without disturbing cells within the cell culture chamber. Cassette 240 also further includes one or more valves 522 or 552, for controlling the flow through the various fluidic pathways (see FIG. 5 for exemplary locations within flowpath).

In exemplary embodiments, as shown in FIG. 2A and FIG. 2B, cell culture chamber 206 is a flat and non-flexible chamber (i.e., made of a substantially non-flexible material such as a plastic) that does not readily bend or flex. The use of a non-flexible chamber allows the cells to be maintained in a substantially undisturbed state. As shown in FIG. 2A, cell culture chamber 206 is oriented so as to allow a cell culture to spread across the bottom of the cell culture chamber. As shown in FIG. 2A, cell culture chamber 206 is suitably maintained in a position that is parallel with the floor or table, maintaining the cell culture in an undisturbed state, allowing the cell culture to spread across a large area of the bottom of the cell culture chamber. In embodiments, the overall thickness of cell culture chamber 206 (i.e., the chamber height) is low, on the order of about 0.5 cm to about 5 cm. Suitably, the cell culture chamber has a volume of between about 0.50 ml and about 300 ml, more suitably between about 50 ml and about 200 ml, or the cell culture chamber has a volume of about 180 ml. The use of a low chamber height (less than 5 cm, suitably less than 4 cm, less than 3 cm, or less then 2 cm) allows for effective media and gas exchange in close proximity to the cells. Ports are configured to allow mixing via recirculation of the fluid without disturbing the cells. Larger height static vessels can produce concentration gradients, causing the area near the cells to be limited in oxygen and fresh nutrients. Through controlled flow dynamics, media exchanges can be performed without cell disturbance. Media can be removed from the additional chambers (no cells present) without risk of cell loss.

As described herein, in exemplary embodiments the cassette is pre-filled with one or more of a cell culture, a culture media, a cell wash media if desired, an activation reagent, and/or a vector, including any combination of these. In further embodiments, these various elements can be added later via suitable injection ports, etc.

As described herein, in embodiments, the cassettes suitably further include one or more of a pH sensor 524, a glucose sensor (not shown), an oxygen sensor 526, a carbon dioxide sensor (not shown), a lactic acid sensor/monitor (not shown), and/or an optical density sensor (not shown). See FIG. 5 for exemplary positions within the flowpath. The cassettes can also include one or more sampling ports and/or injection ports. Examples of such sampling ports 220 and injection ports 222 are illustrated in FIG. 2A, and exemplary locations in the flowpath shown in FIG. 5, and can include an access port for connecting the cartridge to an external device, such as an electroporation unit or an additional media source. FIG. 2A also shows the location of the input 202, prewarming warming bag 224 which can be used to warm cell media, etc., and secondary chamber 230.

In embodiments, cassette 240 can also include a cell wash system 512 that is suitably contained within cassette (i.e., within the structure shown in FIG. 2A), and fluidly connected to the fluidics system of the cassette. In embodiments, cell wash system 512 is a container or bag contained within cassette 240 that suitably includes a cell wash media. The cell wash media is suitably used to clean the desired cell population to remove any undesired waste cells or contamination prior to transferring the cell population within the cassette or outside the cassette for further processing or use. Cell wash system 512 can also be included outside of cassette 102.

Cassette 102 can also further optionally include a cell holding chamber 516 (not visible in FIG. 2 as it is located inside cassette 102). FIG. 5 shows an exemplary location of cell holding chamber 516 in the flowpath for the cassette. Cell holding chamber 516 is suitably a reservoir or suitable chamber located within the cassette into which a cell population can be held, either prior to or following various stages of the processing, as described herein.

In further embodiments, provided herein is a production cassette for use in an automated cell engineering system, comprising: a low temperature chamber including a cell culture media storage vessel and a first array of sealed temperature probes in the cell culture media storage vessel; a high temperature chamber for carrying out activation, transduction and/or expansion of a cell culture, the high temperature chamber separated from the low temperature chamber by a thermal barrier, the high temperature chamber including a cell culture chamber and a second array of sealed temperature probes in the cell culture chamber; one or more fluidics pathways connected to the cell culture chamber and the cell culture media storage vessel, and including a third array of sealed temperature probes in the one or more fluidics pathways; and electric connection elements that are electrically connected to each of the first, second, and third arrays of sealed temperature probes, wherein the one or more fluidics pathways provide recirculation, removal of waste, and homogenous gas exchange and distribution of nutrients to the cell culture chamber.

As noted, the cassettes 240 described herein that contain the sealed temperature probes 252 can be utilized as calibration cassettes and/or production cassettes. In embodiments where the cassettes are utilized as calibration cassettes, the cassettes are designed and implemented as if they were production a cell product or a biomaterial product, but no product is actually produced. Instead the cassette simply provides calibration of the temperature of the associated system/platform, production cassette design and the various sections and regions of the production cassettes, or of a process design for use with the production cassette. In embodiments where the cassette is a production cassette, in addition to providing information regarding the temperature profile within the cassette, the system is also set up to prepare the desired cell and/or biomaterial, for ultimate use in patient or research setting. In embodiments in which the cassette is a production cassette, the temperature probes are suitably removed and cleaned/sterilized or replaced between each use, particularly where each different use is for a different patient.

As described herein, in embodiments of the production cassettes, the cell culture media storage vessel is a bag, and the first array of sealed temperature probes are attached to an internal surface of the bag. Suitably, the cell culture chamber is flat and substantially non-flexible, and the second array of sealed temperature probes in the cell culture chamber are attached to a bottom and/or a side of the cell culture chamber. In additional embodiments, the one or more fluidics pathways include tubing and connectors, and wherein the third array of sealed temperature probes are located within the tubing.

In embodiments, the first, second, and third arrays of sealed temperature probes include resistance temperature detectors (RTD) hermetically sealed by a polymeric cover. Additional examples of temperature probes are described herein. Suitable numbers of probes for use in the various arrays are described herein, and in embodiments, the first, second and third arrays of sealed temperature probes each include at least 2 sealed temperature probes, suitably between 2-4 sealed temperature probes, and in embodiments, the first, second, and third arrays of sealed temperature probes in total include 12 sealed temperature probes.

As described herein, the production cassettes also suitably include a control circuit electrically connected to the electric connection elements for interaction with the temperature probe (i.e., for measurement, recording, modification, etc.). The control circuit can be associated with the production cassette (i.e. connected to the inside or outside of the cassette, or associated with the automated cell engineering system.

The devices, systems and methods described herein are suitably used to monitor, map and/or control the temperature within a cassette of a biomaterial engineering system. However, a similar approach can be utilized with probes that measure other variables of the cassettes, including for example, pH, dissolved oxygen, fluid flow rate, magnetic field, etc. Probes for measuring such variables are known in the art and can be placed within the cassette, similar to the temperature probes, in an array format, allowing for the measurement of these variables over all or a portion of the cassette to map and monitor the variables during the various process steps of the cell engineering or biomaterial engineering methods.

In further embodiments, a remote temperature probe can be used to monitor, record and provide feedback on the temperature of a cassette in an automated biomaterial engineering system. Such remote temperature probe can include, for a example, an infrared temperature detection device that can be mounted within an automated engineering system and record one or more temperatures within he cassette as the automated processes are being carried out.

FIGS. 3A-3B show the COCOON® automated cell engineering system 300 with cassette 240 positioned inside (cover of automated cell engineering system opened in FIG. 3B). Also shown is an exemplary user interface 304, which can include a bar code reader, and the ability to receive using inputs by touch pad or other similar device.

The automated cell engineering systems and cassettes described herein suitably have three relevant volumes, the cell culture chamber volume, the working volume, and the total volume. Suitably, the working volume used in the cassette ranges from 180 mL to 460 mL based on the process step, and can be increased up to about 500 mL, about 600 mL, about 700 mL, about 800 mL, about 900 mL or about 1 L. In embodiments, the cassette can readily achieve 4*10⁹ cells-10*10⁹ cells. The cell concentration during the process varies from 0.3*10⁶ cells/ml to approximately 10*10⁶ cells/ml. The cells are located in the cell culture chamber, but media is continuously recirculated through additional chambers (e.g., crossflow reservoir and satellite volume) to increase the working volume, as described herein.

Fluidics pathways, including gas exchange lines, may be made from a gas-permeable material such as, e.g., silicone. In some embodiments, the automated cell engineering system recirculates oxygen throughout the substantially non-yielding chamber during the cell production methods. Thus, in some embodiments, the oxygen level of a cell culture in the automated cell engineering system is higher than the oxygen level of a cell culture in a flexible, gas-permeable bag. Higher oxygen levels may be important in the cell culture expansion step, as increased oxygen levels may support increased cell growth and proliferation.

FIGS. 3C and 3D depicts embodiments of an automated biological material engineering system, such as the COCOON® automated cell engineering system or some other biological material engineering system, and a cassette that can be placed within the automated biological material engineering system. More specifically, FIG. 3C depicts a cassette 340 having a plurality of temperature probes 352 and having the control circuit 270. The temperature probes 352 may be configured to measure internal temperature within the cassette 340. The figure further depicts an automated biological material engineering system 301 which can receive the cassette 340. The automated biological material engineering system 301 may include a system temperature probe 242 and a heating device or cooling device 250. The system temperature probe 242 may be configured to measure a temperature outside the cassette 340 (which may be referred to as an ambient temperature). In some cases, the automated biological material engineering system 301 may have its own control circuit, which may be separate from the control circuit 270.

FIG. 3D depicts the control circuit 270 being located in or otherwise associated with the automated biological material engineering system 301. In this example, the system 301 may be configured to receive a cassette 341 that has a wireless module 362, and the control circuit 270 of the system 301 may be configured to receive internal temperature measurements from within the cassette 341 via the wireless module 362.

FIG. 3E depicts an example of the cassette 340/341. More particularly, the cassette 340 in this example may have the temperature probes 352 and a printed circuit board for processing temperature measurements made by the temperature probes 352 and/or communicating the temperature measurements to an external device, such as to the control circuit 270 in FIG. 3D. For instance, the printed circuit board may include a frequency filter, an analog front end (AFE), an analog-to-digital converter, and a control circuit (e.g., a microcontroller unit (MCU)) for processing the temperature measurements, and the wireless module 362 for transmitting the temperature measurements to another device. In an embodiment, the MCU may be configured to store the temperature measurements on a removable memory device or some other non-transitory computer-readable or circuit-readable medium. In some cases, the removable memory device and/or in-circuit programming on the printed circuit board may store instructions that can be executed by the MCU.

FIG. 3F depicts an example in which the control circuit 270 is located in or otherwise associated with a computing device 303, such as a laptop computer or desktop computer or other personal computer (PC), which is in communication with the cassette 340/341 and/or the automated biological material engineering system 301. The control circuit 270 may be configured, e.g., to receive temperature measurements or other data from the cassette 340/341 and/or from the automated biological material engineering system 301, and/or may be configured to control one or more components of the automated biological material engineering system 301. In an embodiment, the cassette 340/341/342, the automated biological material engineering system 301, and/or the computing device 302 may include a non-transitory computer-readable medium, such as a hard disk drive (HDD), a solid state drive (SSD), flash memory, or any other storage device. The non-transitory computer-readable medium may store data, such as temperature measurements, and/or instructions which can be executed by the control circuit 270. These instructions may be used to, e.g., perform one or more methods discussed herein, such as the method 600 discussed below.

FIG. 3G illustrates an example in which the automated biological material engineering system 301 may be configured to receive a first cassette or first type of cassette, such as the cassette 340/341, and to receive a second cassette or second type of cassette, such as the cassette 342, either at different times or at the same time. In an embodiment, the cassette 342 may have fewer temperature probes than the cassette 340/341, or may have no temperature probes within the cassette 342. In some cases, the cassette 340/341 may be a calibration cassette, and the cassette 342 may be a production cassette.

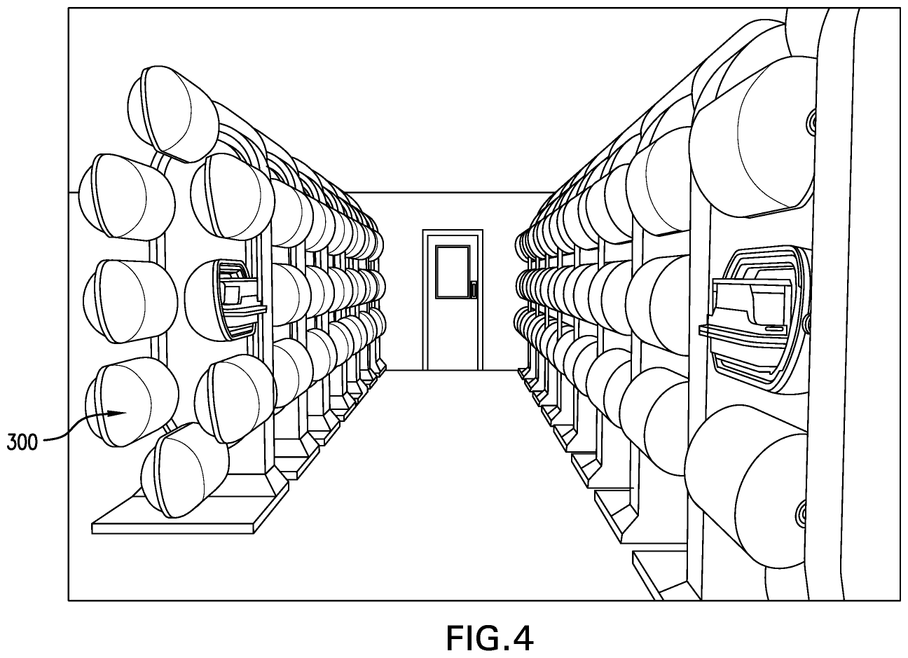

In embodiments, the methods and cartridges described herein are utilized in the COCOON® platform (Octane Biotech (Kingston, ON)), which integrates multiple unit operations in a single turnkey platform. Multiple cell protocols are provided with very specific cell processing objectives. To provide efficient and effective automation translation, the methods described utilize the concept of application-specific/sponsor-specific disposable cassettes that combine multiple unit operations—all focused on the core requirements of the final cell therapy product. Multiple automated cell engineering systems 300 can be integrated together into a large, multi-unit operation for production of large volumes of cells or multiple different cellular samples for individual patients (see FIG. 4).

Also illustrated in FIG. 5 are exemplary positioning of various sensors (e.g., pH sensor 524, dissolved oxygen sensor 526), as well as sampling/sample ports and various valves (including bypass check valves 552), as well as one or more fluidic pathways 540, suitably comprising a silicone-based tubing component, connecting the components. As described herein, use of a silicone-based tubing component allows oxygenation through the tubing component to facilitate gas transfer and optimal oxygenation for the cell culture. Also show in FIG. 5 is the use of one or more hydrophobic filters 554 or hydrophilic filters 556, in the flowpath of the cassette.

In additional embodiments, provided herein is an automated cell engineering system 300. As shown in FIGS. 3A and 3B, automated cell engineering system 300 suitably includes an enclosable housing 302, and cassette 240, contained within the enclosable housing. As used herein, “enclosable housing” refers to a structure than can be opened and closed, and within which cassette 240 as described herein, can be placed and integrated with various components such as fluid supply lines, gas supply lines, power, cooling connections, heating connections, etc. As shown in FIGS. 3A and 3B, enclosable housing can be opened (FIG. 3B) to allow insertion of the cassette, and closed (FIG. 3A) to maintain a closed, sealed environment to allow the various automated processes described herein to take place utilizing the cassette.

Automation of unit operations in cell therapy production provides the opportunity for universal benefits across allogeneic and autologous cell therapy applications. In the unique scenario of patient-specific, autologous cell products, and even more emphasized by the clinical success of these therapies, the advantages of automation are particularly compelling due to the significant micro-lot complexities of small batch GMP compliance, economics, patient traceability and early identification of process deviations. The associated emergence of complex manufacturing protocols draws attention to the fact that the value of end-to-end integration of automated unit operations in micro-lot cell production has not been a point of significant study. However, the expected demand for these therapies following their impending approval indicates that implementation of a fully closed end-to-end system can provide a much needed solution to manufacturing bottlenecks, such as hands-on-time and footprint.

Developers of advanced therapies are encouraged to consider automation early in the rollout of clinical translation and scale up of clinical trial protocols. Early automation can influence protocol development, avoid the need for comparability studies if switching from a manual process to an automated process at a later stage, and provide a greater understanding of the longer-term commercialization route.

Description of Methods

One aspect of the present disclosure relates to methods for performing temperature monitoring and/or temperature control in an automated biological material engineering system, such as the system 300/301 discussed above. The methods may be performed by a control circuit, such as the control circuit 270 discussed above. As stated above, the control circuit 270 may be located in or otherwise associated with a cassette (e.g., cassette 240, 340, or 341 of FIGS. 2B, 2C, 3C, and 3D), located in or otherwise associated with the automated biological material engineering system (e.g., 300/301 of FIGS. 3A-3F), or associated with some other device (e.g., computing device 303).

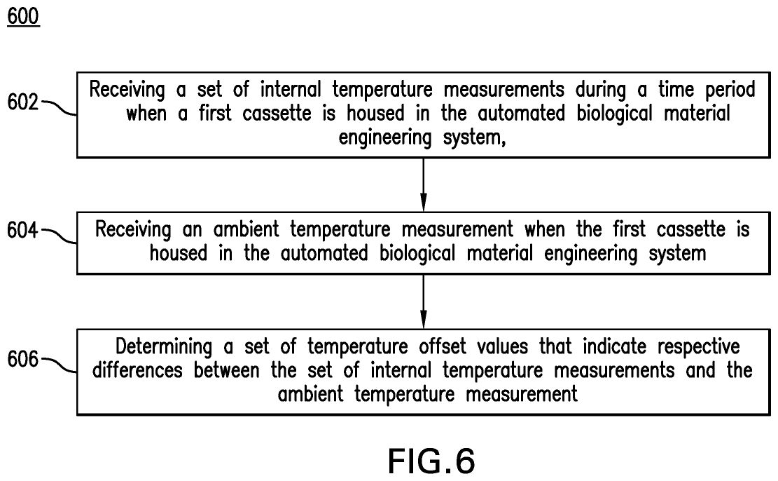

FIG. 6 illustrates a flow diagram for an example method 600 in which temperature monitoring is performed. The method may be performed by, e.g., the control circuit 270. In an embodiment, the method 600 may begin with or otherwise include a step 602, in which the control circuit receives a set of internal temperature measurements that indicate temperature within a first cassette, such as the cassette 240 of FIGS. 2B and 3A or the cassette 340/341 of FIGS. 3D-3F. In an embodiment, the set of internal temperature measurements may be received from an array of temperature probes disposed within the first cassette, such as the array of temperature probes 252 of FIG. 2B or the array of temperature probes 352 of FIGS. 3D-3E (also referred to as temperature sensors). In such an embodiment, the control circuit may receive the set of internal temperature measurements from the array of temperature probes via a wired connection, such as that provided by electrical connection elements 258 in FIG. 2B, or via a wireless connection, such as that provided by the wireless module in FIG. 3D/3E. For instance, if the control circuit (e.g., 270) is disposed within the first cassette, as illustrated in FIG. 3C, the control circuit may in some implementations receive the temperature measurements via a wired connection, such as via the electrical connection elements 258 of FIG. 2A or 2B. If the control circuit (e.g., 270) is disposed outside the first cassette, as illustrated in FIGS. 3D and 3F, the control circuit may receive the temperature measurements via a wireless module 362 or some other communication circuit, or via a wired electrical connection (if any). As stated above, the wireless module 362 may relay data that is collected by a data collection circuit 361 from the temperature probes 352. In some cases, if the temperature probes 352 have an impedance which changes based on temperature, the data collection circuit 361 may be configured to determine impedance values of the temperature probes 352 and to use the impedance values to calculate temperature values. The wireless module 362 may then be configured to wirelessly communicate the temperature values to the control circuit 270. In some cases, if the control circuit 270 has a wired connection to the temperature probes, the control circuit 270 may be configured to calculate the temperature values based on impedance values of the temperature probes.

In some scenarios, the set of internal temperature measurements may be generated by the temperature probes and/or received by the control circuit during a first time period when the first cassette (e.g., 240 or 340/341) is housed in the automated biological material engineering system (e.g., 300/301), such as the scenario illustrated in FIG. 3B. In some implementations, the first cassette may be a calibration cassette, as discussed above, and the first time period may be part of a calibration phase. In some implementations, the first cassette may be a production cassette, which is also discussed above.

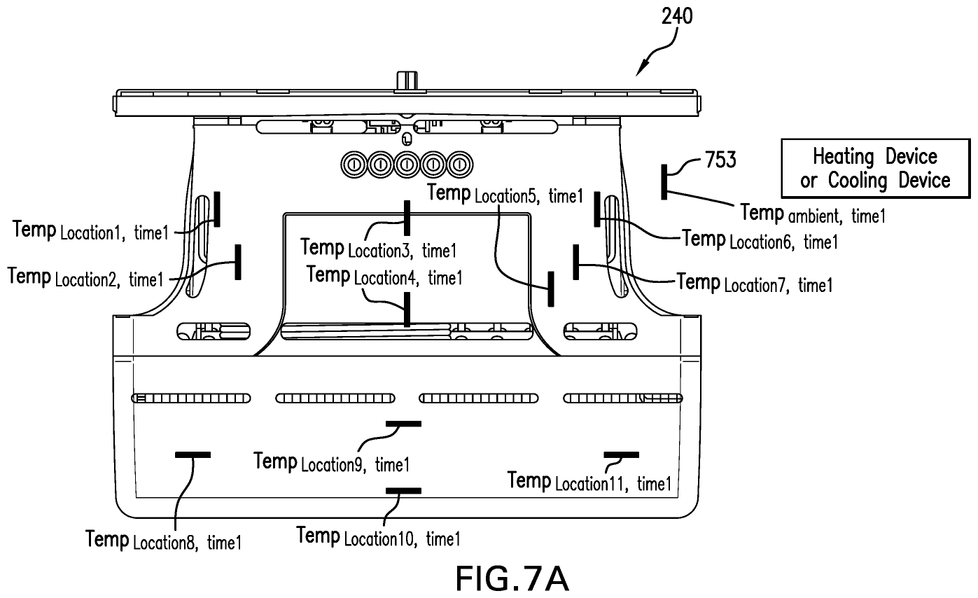

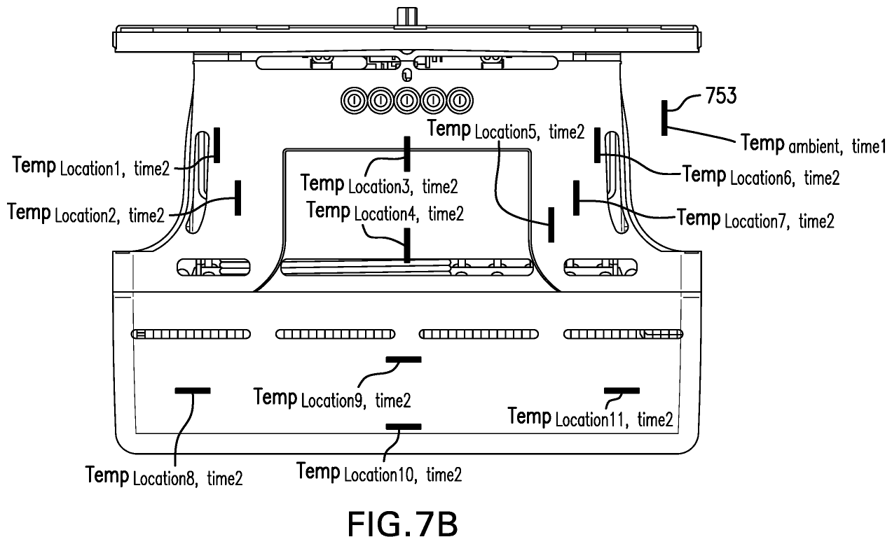

In an embodiment, the array of temperature probes may be disposed at multiple respective locations within the first cassette, as depicted in FIGS. 2B and 2C. In such an embodiment, the set of internal temperature measurements may correspond to the multiple respective locations. For example, FIG. 7A illustrates a set of temperature measurements Temp_{Location1, time1} through Temp_{Location11, time1} that is made at time t₁ (also referred to as time₁). FIG. 7B further illustrates a set of temperature measurements Temp_{Location1, time2} through Temp_{Location11, time2} that is made at time t₂ or time₂. As depicted in FIGS. 7A and 7B, the set of temperature measurements Temp_{Location1}, timer through Temp_{Location11, time1} may indicate temperature at locations 1 through 11 at time t₁ or time₁, and the set of temperature measurements Temp_{Location1, time2} through Temp_{Location11, time2} may indicate temperature at the same locations at time t₂ (also referred to as time₂). In some cases, each of time₁ and time₂ may be a point in time that is during the first time period when the first cassette is disposed within the automated biological material engineering system. The two points in time may, e.g., correspond to two different stages of a biological material production process (e.g., two different steps in the production process) being executed by the biological material engineering system.

In an embodiment, the set of internal temperature measurements may be used by the control circuit to generate a temperature map that indicates how temperature varies spatially across the first cassette. For example, the temperature measurements Temp_{Location1, time1} through Temp_{Location11, time1} may be used to generate a first temperature map which indicates how temperature varies spatially within the first cassette at a first point in time, and the temperature measurements Temp_{Location1, time2} through Temp_{Location11, time2} may be used to generate a second temperature map which indicates how temperature varies spatially within the first cassette at a second point in time.

In an embodiment, the control circuit (e.g., 270) may wirelessly transmit the set of internal temperature measurements to a computing device, such as the computing device 303 in FIG. 3D. The computing device 303 may be, e.g., a desktop computer or laptop that is used to log temperature within the first cassette or within some other cassette compatible with the automated biological material engineering system.

Returning to FIG. 6, the method 600 may in an embodiment include a step 604, in which the control circuit receives an ambient temperature measurement during the time period when the first cassette is housed in the automated biological material engineering system. In an embodiment, the ambient temperature measurement may be generated by a system temperature probe (also referred to as system temperature sensor) of the automated biological material engineering system, such as the system temperature probe 253 illustrated in FIGS. 3C through 3G, or the system temperature probe 753 illustrated in FIGS. 7A and 7B. The system temperature probe may be disposed outside the first cassette, and the ambient temperature measurement may indicate temperature outside the first cassette (e.g., outside cassette 240, 340, or 341). As an example, FIG. 7A depicts an ambient temperature measurement Temp_{ambient,time1} that is generated by the system temperature probe 753 at time₁ and/or received by the control circuit at a first point in time, namely time₁, while FIG. 7B depicts an ambient temperature measurement Temp_{ambient,time2} that is generated by the probe 753 and/or received by the control circuit at a second point in time, namely time₂.

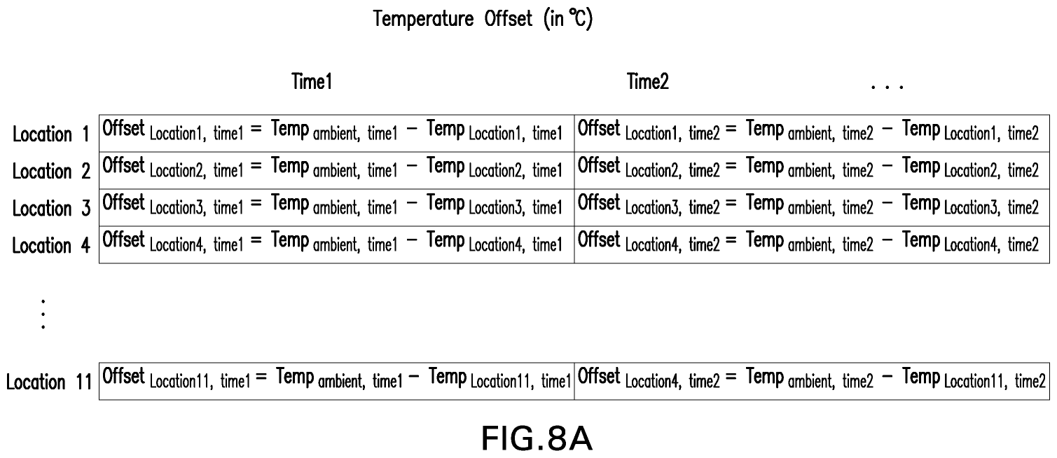

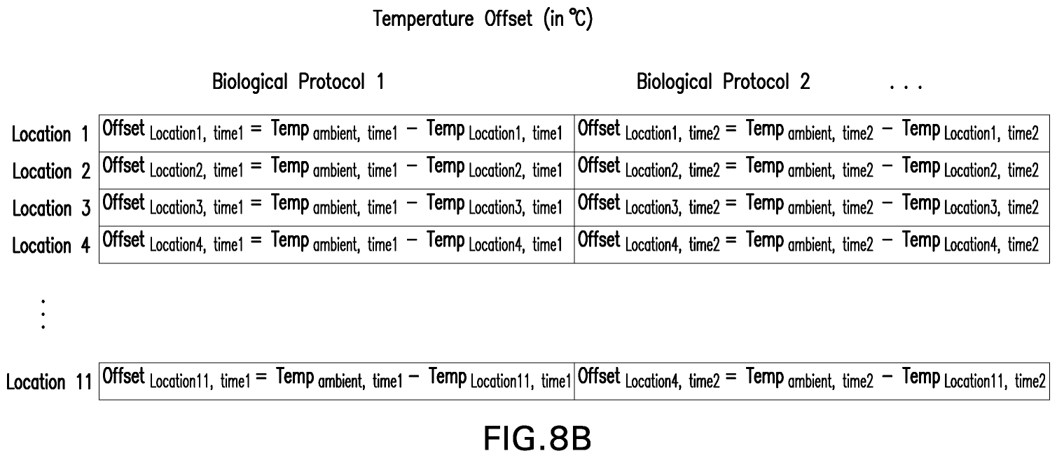

Returning to FIG. 7B, the method 600 may in an embodiment include a step 606, in which the control circuit determines a set of temperature offset values that indicate respective differences between the set of internal temperature measurements and the ambient temperature measurement. For example, FIG. 8A provides an example of a first set of temperature offset values that indicate respective differences between the first set of internal temperature measurements Temp_{Location1, time1} through Temp_{Location11, time1} corresponding to a first point in time and the ambient temperature measurement Temp_{ambient,time1} corresponding to the first point in time. The figure provides an example of a second set of temperature offset values that indicate respective differences between the second set of internal temperature measurements Temp_{Location1, time2} through Temp_{Location11, time2}, which correspond to a second point in time, and the ambient temperature measurement Temp_{ambient,time2}, which also correspond to the second point in time. In some cases, the first point in time may correspond to a first stage of a biological material production process (e.g., a first biological protocol), while the second point in time may correspond to a second stage of a biological material production process (e.g., a second biological protocol), as illustrated in FIG. 8B. As FIGS. 8A and 8B illustrate, the temperature offset values that are determined in step 606 may in an embodiment be dynamic offset values that cover different biological protocols, or more generally cover different points in time in the first time period.

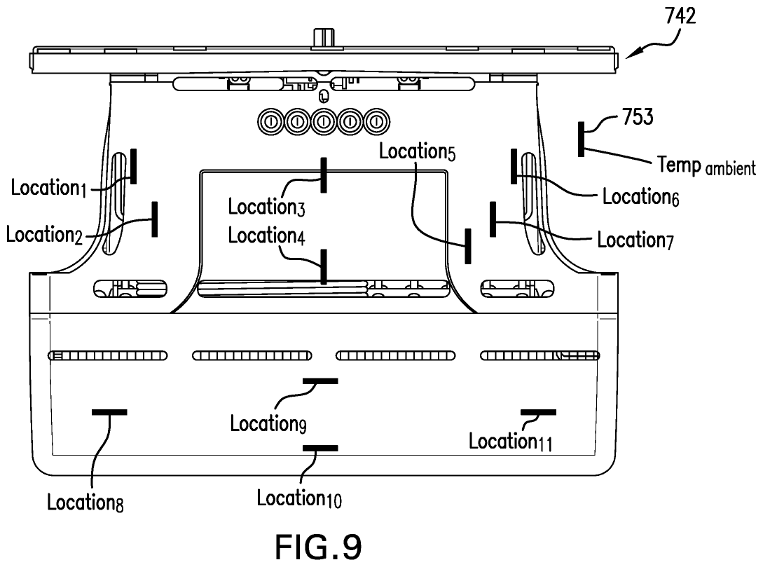

In an embodiment, the temperature offset values may be used to facilitate temperature control. The temperature control may involve, e.g., controlling a heating device or a cooling device, such as device 250 in FIGS. 3C and 3D, to cause a location in a cassette, such as cassette 342 in FIG. 3G or a cassette 742 in FIG. 9, to reach a desired temperature, such as a target temperature value. In some cases, the cassette (e.g., 240 or 340/341) used to determine temperature offset values, which is discussed above, may be a first cassette, such as a calibration cassette. In this embodiment, the cassette (e.g., 342/742) for which the temperature control is performed may be a second cassette, such as a production cassette. In such cases, the temperature offset values may have been determined during a first time period, in which the first cassette is disposed within the automated biological engineering system, and the temperature control may be performed during a second time period, in which the second cassette is disposed within the automated biological engineering system. During the second time period, the first cassette may be, e.g., taken out from the automated biological material engineering system, while the second cassette may be placed into the system. In some implementations, the second cassette may have no temperature probes disposed therewithin, or may have fewer temperature probes relative to how many temperature probes are disposed in the first cassette.

In an embodiment, the determination of the temperature offset values and the controlling of temperature may be performed by the same control circuit, or may be performed by two separate control circuits. For example, a first control circuit may determine the temperature offset values and store them in a storage device, while the same control circuit or another control circuit may later retrieve the temperature offset values from the storage device and perform temperature control based on the temperature offset values.

In an embodiment, performing the temperature control may involve using the temperature offset values determined in step 606 to determine a relationship between an ambient temperature value and an internal temperature value at a location within the cassette (e.g., 342/742). In some cases, although the automated biological material engineering system may have a system temperature probe, such as probe 753 in FIG. 9, to determine the ambient temperature value, the cassette (e.g., 342/742) may have no temperature probe, or only a few temperature probes. Thus, the control circuit may have no direct measurement, or only a few direct measurements regarding internal temperature within the cassette (e.g., 342/742). In such cases, the control circuit (e.g., 270) may rely on the temperature offset values to infer information about temperature within the cassette (e.g., 342/742) based on the ambient temperature value measured by the system temperature probe, and/or to determine what ambient temperature to target in order to bring about a desired internal temperature for a location within the cassette (e.g., 342/742).

In an embodiment, the control circuit which is performing temperature control (e.g., control circuit 270) may determine the target internal temperature value for a location in a cassette, such as the second cassette (e.g., 342/724) discussed above. The determination may be performed before or after the cassette is placed within an automated biological material engineering system. In some cases, the location may be one of Locations 1 through 11 in the cassette 742 of FIG. 9. These locations may be the same or otherwise correspond to respective locations at which Temp_{Location1} through Temp_{Location11} were measured in the cassette 240 of FIGS. 7A and 7B. In some cases, the location associated with the target internal temperature value may be a location between two or more of Locations 1 through 11 in FIG. 9. In some cases, if the automated biological material engineering system is an automated cell engineering system, the target internal temperature value may be a desired cell culture temperature value for a cell culture within the cassette (e.g., 342/742). The desired cell culture temperature value may be a value that, e.g., promotes cell growth.

In an embodiment, the control circuit (e.g., 270) may control temperature by controlling a heating device or cooling device of the automated biological material engineering system. This operation may occur during a time period in which the cassette (e.g., 342/742) is disposed within the automated biological material engineering system. As stated above, this time period may be a second time period, while the set of temperature offset values may have been determined during a first time period when another cassette (e.g., 240) is disposed within the automated biological material engineering system, as discussed above.

In an embodiment, the control circuit may control the heating device or cooling device based on the target internal temperature value, the set of temperature offset values, and one or more ambient temperature measurements generated by a system temperature probe (e.g., 753). More specifically, the control circuit may control the heating device or the cooling device to cause an estimated internal temperature value to reach the target internal temperature value, and/or to cause the an ambient temperature value to reach a target ambient temperature value. The estimated internal temperature value and/or the target ambient temperature value may be determined based on the set of temperature offset values, as discussed below in more detail.

In an embodiment, the control circuit may perform temperature control by estimating an internal temperature value for a location in the cassette (e.g., 342/742), so that the control circuit can control the heating device or cooling device based on a difference between the estimated internal temperature value and the target internal temperature value, or more specifically to cause the difference to decrease so that the estimated internal temperature value approaches the target internal temperature value, as stated above. In some cases, the estimated internal temperature value for the location (e.g., 342/742) in the cassette may be estimated based on an ambient temperature value measured by a system temperature probe (e.g., 753) in FIG. 9, and based on a temperature offset value corresponding to that location in the cassette (e.g., 342/742). The temperature offset value for the location in the cassette (e.g., 342/742), such as the second cassette discussed above, may be equal to or based on one of the set of temperature offset values discussed above with respect to step 606. The set of temperature offset values may have been determined using another cassette, such as the first cassette discussed above (e.g., 240 or 340/341). More specifically, the temperature offset value for a location in the second cassette may be equal to or based on a temperature offset value for a corresponding location in the first cassette. In some implementations, the first cassette and the second cassette may have a same or similar shape or layout, and a location in the first cassette may correspond to a location in the second cassette if the two locations are identically or similarly situated spatially relative to their respective cassettes. That is, the two corresponding locations may be identical or similar locations relative to their respective cassettes.

As an example, Location 1 in the cassette 240 of FIGS. 7A and 7B (at which Temp_{Location1, time1} and Temp_{Location1, time2} are measured) may correspond to Location 1 in the cassette 742 of FIG. 9. In such an example, the temperature offset value for Location 1 in the cassette 742 may be equal to or based on a temperature offset value for a corresponding location in the cassette 240, such as Offset_{Location1,time1} or Offset_{Location1,time2}, which may have been determined in step 606. The control circuit may thus determine the estimated internal temperature value for Location 1 in the cassette 742 based on, e.g., the ambient temperature value, such as Temp ambient in FIG. 9, and based on Offset_{Location1,time1} or Offset_{Location1,time2}. For instance, the control circuit may determine the estimated internal temperature value for Location 1 in the cassette 742 by subtracting Offset_{Location1,time1} or Offset_{Location1,time2} from the ambient temperature value.

As stated above, the temperature offset values determined in step 606 may, in an embodiment, be dynamic offset values which account for various points in time (e.g., various biological protocols) within a time period, such as the first time period discussed above in which the first cassette (e.g., 240) is disposed within the automated biological material engineering system. For instance, as depicted in FIGS. 8A and 8B, the dynamic temperature offset values determined during the first time period may be organized into multiple sets of temperature offset values that correspond to different points in time during the first time period, such as a first set of temperature offset values Offset_{Location1,time1} through Offset_{Location11,time1} corresponding to times (e.g., a first biological protocol) and a second set of temperature offset values Offset_{Location1,time2} through Offset_{Location11,time2} corresponding to time₂ (e.g., a second biological protocol). In this embodiment, determining the estimated internal temperature value for a location in the second cassette (e.g., 342/742) for a point in time in the second time period (in which the second cassette is located in the automated biological material engineering system) may involve determining a temperature offset value which corresponds to that point in time, as well as to that location. The point in time may be when the internal temperature value is being estimated or when the estimated value is going to be used.

In some instances, the corresponding temperature offset value may be a value which was estimated at a corresponding point in time during the first time period discussed above (when the multiple sets of temperature offset values are determined). In these instances, a point in time in the first time period may correspond to a point in time in the second time period if, e.g., the two points in time belong to a same stage of a biological material production process (e.g., belong to a same biological protocol), and/or if they have a same temporal offset relative to a beginning or end of their respective time periods. As an example, if the control circuit is determining the estimated internal temperature value for Location 1 during a first biological protocol in the second time period, then the control circuit may determine that the corresponding temperature offset value is Offset_{Location1,time1}, which corresponds to Location 1 and corresponds to the first biological protocol. If the control circuit is determining the estimated internal temperature value for the same location during a second biological protocol in the second time period, then the control circuit may determine that the corresponding temperature offset value is Offset_{Location1,time2}.

In some cases, the control circuit may perform temperature control for the cassette (e.g., 342/742) discussed above by estimating a temperature map which indicates how temperature varies spatially across the cassette. The temperature map may be generated based on temperature offset values, such as the first set and/or the second set of temperature offset values discussed above, and one or more temperature ambient measurements generated during the second time period. In some implementations, the control circuit may generate the temperature map by estimating internal temperature values at various locations within the cassette, as discussed above. For instance, the control circuit may subtract, from the ambient temperature measurement (or, more specifically from the ambient temperature value), temperature offset values which correspond to those locations and correspond to a point in time at which the ambient temperature measurement is made.

In some implementations, the control circuit may determine the temperature map for the cassette (e.g., 342/742) in second time period based on the temperature map generated in the first time period. In the above implementations, the temperature map may indicate estimated internal temperature values at various locations in the cassette (e.g., 342/742) at a point in time, and the control circuit may control the heating device or the cooling device based on the temperature map. The control circuit may be configured to generate a single temperature map, or may generate multiple temperature maps corresponding to multiple points in time within the second time period (e.g., corresponding to multiple biological protocols). If the control circuit is determining the temperature map for the first point in time within the second time period, it may select the first set of temperature offset values Offset_{Location1,time1} through Offset_{Location11,time1} to determine the temperature map. If the control circuit is determining the temperature map for the second point in time within the second time period, it may select the second set of temperature offset values Offset_{Location1,time2} through Offset_{Location11,time2} to determine the temperature map.

As stated above, the control circuit may perform the heating control by causing an estimated internal temperature value to approach a target internal temperature value, and/or by causing a measured ambient temperature value to approach a target ambient temperature value. In an embodiment, the control circuit may determine the target ambient temperature value based on the target internal temperature value and/or the estimated internal temperature value. For instance, the control circuit may determine the target ambient temperature value by adding a target internal temperature value for a particular location within the cassette to temperature offset value corresponding to that location. As an example, the control circuit may determine the target ambient temperature value by adding a target internal temperature value for Location 7 in FIG. 9 to Offset_{Location7,time1} or Offset_{Location7,time2}.

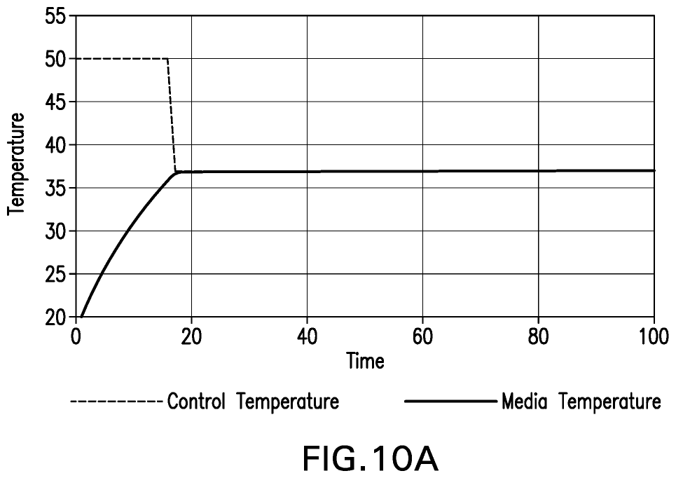

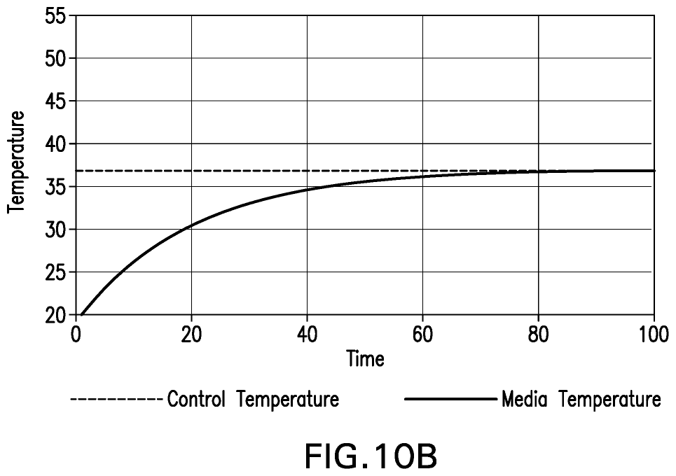

In an embodiment, the control circuit may determine multiple target ambient temperature values for multiple points in the second time period. For instance, to cause Location 7 in the cassette (e.g., 742) to reach a target internal temperature value(s) at various points in time, the control circuit may determine a first target ambient temperature value for a first point in time within the second time period by using Offset_{Location7,time1}, which corresponds to the first point in time. The control circuit may determine a second target ambient temperature value for a second point within the second time period by using Offset_{Location7,time2}, which corresponds to the second point in time. The two points in time may be part of two different biological protocols within the second time period, or may be part of the same biological protocol. The control circuit may, e.g., control (e.g., activate or deactivate) the heating device or the cooling device to cause the measured ambient temperature, which may be measured by the system temperature probe (e.g., 753), to transition from the first target ambient temperature value to the second target ambient temperature value. For example, FIG. depicts an example of a target ambient temperature and/or measured ambient temperature, which may be referred to as a control temperature because it is used to control a heating device or cooling device. The figure further depicts an internal temperature value, which may be referred to as media temperature because the cassette in this example may contain a cell culture media. In this example, the control circuit may control a heating device to cause the control temperature to transition from a first target ambient temperature value to a second ambient temperature value, or more specifically from a higher target ambient temperature value to a lower ambient temperature value. The use of at least two different target ambient temperature values may cause a quicker heating or cooling of the media within the cassette. For instance, the use of the higher initial ambient temperature value in FIG. 10A may cause the internal temperature value (e.g., media temperature) to rise more quickly relative to an implementation in which the control circuit simply tries to maintain a single target ambient temperature or control temperature throughout a particular time period. The latter example, in which a single control temperature is used, is depicted in FIG. 10B. Thus, the example in FIG. 10A may use the heating device in a more aggressive and/or dynamic manner to better compensate for a thermal lag between the ambient temperature and the internal temperature (e.g., media temperature), and to more quickly bring the internal temperature to a target value.

In an embodiment, the temperature offset values may facilitate the more aggressive and/or dynamic manner of controlling the heating device or cooling device. More particularly, although raising the ambient temperature may help compensate for the thermal lag between the ambient temperature and the internal temperature, the control circuit may need to ensure that the internal temperature does not become excessively high or excessively low, which may damage e.g., a cell culture media in the cassette discussed above (e.g., 342/742). The control circuit may use the temperature offset values to more accurately determine an ambient temperature value which is not likely to cause the internal temperature value to become excessively high or excessively low, and/or to more accurately estimate the internal temperature value to confirm that it is not excessively high or excessively low, even if the control circuit cannot directly measure the internal temperature value. In an embodiment, if the temperature offset values are dynamic by corresponding to multiple points in time, then the control circuit may use the dynamic temperature offset values to make adjustments to how the ambient temperature is being controlled. For example, the dynamic temperature offset values may aid the control circuit in determining when to transition from the higher initial ambient temperature value (e.g., target ambient temperature value) to the lower ambient temperature of FIG. 10A, so as to quickly raise the internal temperature but also avoid causing the internal temperature to overshoot its target value.

In an embodiment, the automated material engineering system has multiple system temperature probes that measure multiple ambient temperature values at multiple respective locations outside of a cassette (e.g., 342/742), the control circuit may be configured to determine multiple target ambient temperature values corresponding to the multiple system temperature probes and/or to the multiple locations.

### Additional Exemplary Embodiments

Embodiment 1 relates to a calibration cassette for use in an automated biological material engineering system. The calibration cassette in this embodiment comprises a low temperature chamber including a media storage vessel and a first array of sealed temperature probes in the media storage vessel; a high temperature chamber separated from the low temperature chamber by a thermal barrier, the high temperature chamber including a cell culture chamber and a second array of sealed temperature probes in the cell culture chamber; one or more fluidics pathways connected to the cell culture chamber and the media storage vessel, and including a third array of sealed temperature probes in the one or more fluidics pathways; and electrical connection elements that are electrically connected to each of the first, second, and third arrays of sealed temperature probes.

Embodiment 2 includes the calibration cassette of embodiment 1, wherein the media storage vessel is a bag, and wherein the first array of sealed temperature probes are attached to an internal surface of the bag.

Embodiment 3 includes the calibration cassette of embodiment 1 or 2, wherein the cell culture chamber is flat and substantially non-flexible, and wherein the second array of sealed temperature probes are attached to a bottom and/or a side of the cell culture chamber.

Embodiment 4 includes the calibration cassette of any one of embodiments 1-3, wherein the one or more fluidics pathways include tubing and connectors, and wherein the third array of sealed temperature probes are located within the tubing.

Embodiment 5 includes the calibration cassette of any one of embodiments 1-4, wherein the first, second, and third arrays of sealed temperature probes include resistance temperature detectors (RTD) hermetically sealed by a polymeric cover.

Embodiment 6 includes the calibration cassette of any one of embodiments 1-5, wherein the first, second, and third arrays of sealed temperature probes each include at least 2 sealed temperature probes.

Embodiment 7 includes the calibration cassette of embodiment 6, wherein the first, second, and third arrays of sealed temperature probes each include between 2-4 sealed temperature probes.

Embodiment 8 includes the calibration cassette of embodiment 7, wherein the first, second, and third arrays of sealed temperature probes in total include 12 sealed temperature probes.

Embodiment 9 includes the calibration cassette of any one of embodiments 1-8, wherein the electric connection elements are electrically connected to a control circuit associated with the calibration cassette.

Embodiment 10 includes the calibration cassette of any one of embodiments 1-8, wherein the electric connection elements are configured to be electrically connected to a control circuit associated with the automated biological material engineering system.

Embodiment 11 relates to a production cassette for use in an automated cell engineering system. The production cassette in this embodiment includes a low temperature chamber including a cell culture media storage vessel and a first array of sealed temperature probes in the cell culture media storage vessel; a high temperature chamber for carrying out activation, transduction and/or expansion of a cell culture, the high temperature chamber separated from the low temperature chamber by a thermal barrier, the high temperature chamber including a cell culture chamber and a second array of sealed temperature probes in the cell culture chamber; one or more fluidics pathways connected to the cell culture chamber and the cell culture media storage vessel, and including a third array of sealed temperature probes in the one or more fluidics pathways; and electric connection elements that are electrically connected to each of the first, second, and third arrays of sealed temperature probes, wherein the one or more fluidics pathways provide recirculation, removal of waste, and homogenous gas exchange and distribution of nutrients to the cell culture chamber.

Embodiment 12 includes the production cassette of embodiment 11, wherein the cell culture media storage vessel is a bag, and wherein the first array of sealed temperature probes are attached to an internal surface of the bag.

Embodiment 13 includes the production cassette of embodiment 11 or 12, wherein the cell culture chamber is flat and substantially non-flexible, and wherein the second array of sealed temperature probes in the cell culture chamber are attached to a bottom and/or a side of the cell culture chamber.

Embodiment 14 includes the production cassette of any one of embodiments 11-13, wherein the one or more fluidics pathways include tubing and connectors, and wherein the third array of sealed temperature probes are located within the tubing.

Embodiment 15 includes the production cassette of any one of embodiments 11-14, wherein the first, second, and third arrays of sealed temperature probes include resistance temperature detectors (RTD) hermetically sealed by a polymeric cover.

Embodiment 16 includes the production cassette of any one of embodiments 11-15, wherein the first, second and third arrays of sealed temperature probes each include at least 2 sealed temperature probes.

Embodiment 17 includes the production cassette of embodiment 16, wherein the first, second, and third arrays of sealed temperature probes each include between 2-4 sealed temperature probes.

Embodiment 18 includes the production cassette of embodiment 17, wherein the first, second, and third arrays of sealed temperature probes in total include 12 sealed temperature probes.

Embodiment 19 includes the production cassette of any one of embodiments 11-18, wherein the electric connection elements are electrically connected to a control circuit associated with the production cassette.

Embodiment 20 includes the production cassette of any one of embodiments 11-18, wherein the electric connection elements are configured to be connected to a control circuit associated with the automated cell engineering system.

Embodiment 21 includes a method of temperature monitoring in an automated biological material engineering system. The method in this embodiment comprises receiving, by a control circuit, a set of internal temperature measurements during a time period when a first cassette is housed in the automated biological material engineering system, wherein the set of internal temperature measurements indicate temperature within the first cassette, and are generated during the time period by an array of temperature probes disposed within the first cassette; receiving, by the control circuit, an ambient temperature measurement when the first cassette is housed in the automated biological material engineering system, wherein the ambient temperature measurement indicates temperature outside the first cassette, and is generated during the time period by a system temperature probe of the automated biological material engineering system that is disposed outside the first cassette; and determining, by the control circuit, a set of temperature offset values that indicate respective differences between the set of internal temperature measurements and the ambient temperature measurement.

Embodiment 22 includes the method of embodiment 21, wherein the control circuit is associated with the first cassette.

Embodiment 23 includes the method of embodiment 21, wherein the control circuit is associated with the automated biological material engineering system.

Embodiment 24 includes the method of any one of embodiments 21-23, wherein the automated biological material engineering system is an automated cell engineering system.

Embodiment 25 includes the method of any one of embodiments 21-24, wherein the array of temperature probes is disposed at multiple respective locations within the first cassette, and the set of internal temperature measurements corresponds to the multiple respective locations within the first cassette, wherein the method further comprises: generating, based on the set of internal temperature measurements, a temperature map that indicates how temperature varies spatially across the first cassette.

Embodiment 26 includes the method of embodiment 25, wherein the set of internal temperature measurements is a first set of internal temperature measurements corresponding to a first point in time within the time period, and wherein the temperature map is a first temperature map that indicates how temperature varies spatially across the first cassette at the first point in time within the time period, wherein the method further comprises: receiving a second set of internal temperature measurements generated by the array of temperature probes in the first cassette, wherein the second set of internal temperature measurements indicate temperature at the multiple respective locations at a second point in time within the time period; and generating, based on the second set of internal temperature measurements, a second temperature map that indicates how temperature varies spatially across the first cassette at the second point in time within the time period.

Embodiment 27 includes the method of embodiment 26, wherein the ambient temperature measurement is a first ambient temperature measurement corresponding to the first point in time within the time period, and the set of temperature offset values is a first set of temperature offset values also corresponding to the first point in time, wherein the method further comprises: receiving a second ambient temperature measurement that indicates temperature outside the first cassette at the second point in time within the time period; and determining a second set of temperature offset values that indicate respective differences between the second ambient temperature measurement and the second set of internal temperature measurements, wherein the second set of temperature offset values correspond to the second point in time, wherein the first point in time belongs to a first stage of a biological material production process, and the second point in time belongs to a second stage of a biological material production process.

Embodiment 28 includes the method of any one of embodiments 21-27, further comprising wirelessly transmitting the set of internal temperature measurements to a computing device.

Embodiment 29 includes relates to a method of temperature control performed in automated biological material engineering system, the method comprising: receiving, by a control circuit, a set of internal temperature measurements during a first time period when a first cassette is housed in the automated biological material engineering system, wherein the set of internal temperature measurements indicate temperature within the first cassette, and are generated during the first time period by an array of temperature probes disposed within the first cassette; receiving, by the control circuit, a first ambient temperature measurement when the first cassette is housed in the automated biological material engineering system, wherein the first ambient temperature measurement indicates temperature outside the first cassette, and is generated during the first time period by a system temperature sensor of the automated biological material engineering system that is disposed outside the first cassette; determining, by the control circuit, a set of temperature offset values that indicate respective differences between the set of internal temperature measurements and the first ambient temperature measurement; determining, by the control circuit, a target internal temperature value for a location in a second cassette; and controlling, by the control circuit during a second time period when the second cassette is housed in the automated biological material engineering system, a heating device or cooling device of the automated biological material engineering system based on the target internal temperature value, the set of temperature offset values, and one or more additional ambient temperature measurement generated by the system temperature sensor during the second time period, wherein the system temperature sensor is disposed outside the second cassette.

Embodiment 30 includes the method of embodiment 29, wherein the automated biological material engineering system is an automated cell engineering system, and the target internal temperature value is a desired cell culture temperature value for a cell culture within the second cassette.

Embodiment 31 includes the method of embodiment 29 or 30, further comprising generating, based on the set of temperature offset values and the one or more additional ambient temperature measurements generated during the second time period, a temperature map that indicates how temperature varies spatially across the second cassette, wherein the heating device or cooling device is controlled based on the temperature map.

Embodiment 32 includes the method of embodiment 31, wherein controlling the heating device or cooling device comprises determining an estimated internal temperature value for the location in the second cassette, wherein the heating device or the cooling device is controlled based on a difference between the estimated internal temperature value and the desired internal temperature value.

Embodiment 33 includes the method of embodiment 32, wherein controlling the heating device or the cooling device comprises determining a target ambient temperature value based on the estimated internal temperature value and/or the target internal temperature value, wherein the heating device or the cooling device is controlled to cause temperature measured by the system temperature probe to approach the target ambient temperature value.

Embodiment 34 includes the method of embodiment 33, wherein the set of temperature offset values is one of multiple sets of temperature offset values determined during the first time period, wherein the multiple sets correspond to different points in time within the first time period, wherein the determined temperature map corresponds to a point in time within the second time period, and is generated by selecting the set of temperature offset values from among the multiple sets of temperature offset values based on a determination that the set of temperature offset values also corresponds to the point in time within the second time period.

Embodiment 35 includes the method of embodiment 34, wherein the set of temperature offset values is associated with a point in time within the first time period, and wherein the point in time within the first time period and the point in time within the second time period both belong to a same stage of a biological material production process that has multiple stages.

Embodiment 36 includes the method of embodiment 34 or 35, wherein the temperature map is a first temperature map, the method further comprising: selecting, at a second point in time within the second time period, a second set of temperature offset values from among the multiple sets of temperature offset values, wherein the selected second set of temperature offset values correspond to the second point in time within the second time period; generating a second temperature map based on the second set of temperature offset values, wherein the second temperature map is associated with the second point in time within the second time period, and wherein the heating device or the cooling device is controlled based on the second temperature map.

Embodiment 37 includes the method of embodiment 36, wherein the target ambient temperature value is a first target ambient temperature value, the method further comprising: determining a second estimated internal temperature value determining a second target ambient temperature value based on the second estimated internal temperature value and/or the target internal temperature value; and controlling the heating device or the cooling device to cause temperature measured by the system temperature probe to transition from the first target ambient temperature value to the second target ambient temperature value.

Embodiment 38 includes the method of embodiment 37, wherein the first target ambient temperature value is higher than the second target ambient temperature value.

Embodiment 39 includes the method of any one of embodiment 21-38, wherein the second cassette has no temperature sensor disposed therein.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A calibration cassette for use in an automated biological material engineering system, comprising:
a low temperature chamber including a media storage vessel and a first array of sealed temperature probes in the media storage vessel;
a high temperature chamber separated from the low temperature chamber by a thermal barrier, the high temperature chamber including a cell culture chamber and a second array of sealed temperature probes in the cell culture chamber;
one or more fluidics pathways connected to the cell culture chamber and the media storage vessel, and including a third array of sealed temperature probes in the one or more fluidics pathways; and
electrical connection elements that are electrically connected to each of the first, second, and third arrays of sealed temperature probes.

2. The calibration cassette of claim 1, wherein the media storage vessel is a bag, and wherein the first array of sealed temperature probes are attached to an internal surface of the bag.

3. The calibration cassette of claim 1 or claim 2, wherein the cell culture chamber is flat and substantially non-flexible, and wherein the second array of sealed temperature probes are attached to a bottom and/or a side of the cell culture chamber.

4. The calibration cassette of any one of claims 1-3, wherein the one or more fluidics pathways include tubing and connectors, and wherein the third array of sealed temperature probes are located within the tubing.

5. The calibration cassette of any one of claims 1-4, wherein the first, second, and third arrays of sealed temperature probes include resistance temperature detectors (RTD) hermetically sealed by a polymeric cover.

6. The calibration cassette of any one of claims 1-5, wherein the first, second, and third arrays of sealed temperature probes each include at least 2 sealed temperature probes.

7. The calibration cassette of claim 6, wherein the first, second, and third arrays of sealed temperature probes each include between 2-4 sealed temperature probes.

8. The calibration cassette of claim 7, wherein the first, second, and third arrays of sealed temperature probes in total include 12 sealed temperature probes.

9. The calibration cassette of any one of claims 1-8, wherein the electric connection elements are electrically connected to a control circuit associated with the calibration cassette.

10. The calibration cassette of any one of claims 1-8, wherein the electric connection elements are configured to be electrically connected to a control circuit associated with the automated biological material engineering system.

11. A production cassette for use in an automated cell engineering system, comprising:
a low temperature chamber including a cell culture media storage vessel and a first array of sealed temperature probes in the cell culture media storage vessel;
a high temperature chamber for carrying out activation, transduction and/or expansion of a cell culture, the high temperature chamber separated from the low temperature chamber by a thermal barrier, the high temperature chamber including a cell culture chamber and a second array of sealed temperature probes in the cell culture chamber;
one or more fluidics pathways connected to the cell culture chamber and the cell culture media storage vessel, and including a third array of sealed temperature probes in the one or more fluidics pathways; and
electric connection elements that are electrically connected to each of the first, second, and third arrays of sealed temperature probes,
wherein the one or more fluidics pathways provide recirculation, removal of waste, and homogenous gas exchange and distribution of nutrients to the cell culture chamber.

12. The production cassette of claim 11, wherein the cell culture media storage vessel is a bag, and wherein the first array of sealed temperature probes are attached to an internal surface of the bag.

13. The production cassette of claim 11 or claim 12, wherein the cell culture chamber is flat and substantially non-flexible, and wherein the second array of sealed temperature probes in the cell culture chamber are attached to a bottom and/or a side of the cell culture chamber.

14. The production cassette of any one of claims 11-13, wherein the one or more fluidics pathways include tubing and connectors, and wherein the third array of sealed temperature probes are located within the tubing.

15. The production cassette of any one of claims 11-14, wherein the first, second, and third arrays of sealed temperature probes include resistance temperature detectors (RTD) hermetically sealed by a polymeric cover.

16. The production cassette of any one of claims 11-15, wherein the first, second and third arrays of sealed temperature probes each include at least 2 sealed temperature probes.

17. The production cassette of claim 16, wherein the first, second, and third arrays of sealed temperature probes each include between 2-4 sealed temperature probes.

18. The production cassette of claim 17, wherein the first, second, and third arrays of sealed temperature probes in total include 12 sealed temperature probes.

19. The production cassette of any one of claims 11-18, wherein the electric connection elements are electrically connected to a control circuit associated with the production cassette.

20. The production cassette of any one of claims 11-18, wherein the electric connection elements are configured to be connected to a control circuit associated with the automated cell engineering system.

21. A method of temperature monitoring in an automated biological material engineering system, the method comprising:
receiving, by a control circuit, a set of internal temperature measurements during a time period when a first cassette is housed in the automated biological material engineering system, wherein the set of internal temperature measurements indicate temperature within the first cassette, and are generated during the time period by an array of temperature probes disposed within the first cassette;
receiving, by the control circuit, an ambient temperature measurement when the first cassette is housed in the automated biological material engineering system, wherein the ambient temperature measurement indicates temperature outside the first cassette, and is generated during the time period by a system temperature probe of the automated biological material engineering system that is disposed outside the first cassette; and
determining, by the control circuit, a set of temperature offset values that indicate respective differences between the set of internal temperature measurements and the ambient temperature measurement.

22. The method of claim 21, wherein the control circuit is associated with the first cassette.

23. The method of claim 21, wherein the control circuit is associated with the automated biological material engineering system.

24. The method of any one of claims 21-23, wherein the automated biological material engineering system is an automated cell engineering system.

25. The method of any one of claims 21-24, wherein the array of temperature probes is disposed at multiple respective locations within the first cassette, and the set of internal temperature measurements corresponds to the multiple respective locations within the first cassette, wherein the method further comprises:
generating, based on the set of internal temperature measurements, a temperature map that indicates how temperature varies spatially across the first cassette.

26. The method of claim 25, wherein the set of internal temperature measurements is a first set of internal temperature measurements corresponding to a first point in time within the time period, and wherein the temperature map is a first temperature map that indicates how temperature varies spatially across the first cassette at the first point in time within the time period, wherein the method further comprises:
receiving a second set of internal temperature measurements generated by the array of temperature probes in the first cassette, wherein the second set of internal temperature measurements indicate temperature at the multiple respective locations at a second point in time within the time period; and
generating, based on the second set of internal temperature measurements, a second temperature map that indicates how temperature varies spatially across the first cassette at the second point in time within the time period.

27. The method of claim 26, wherein the ambient temperature measurement is a first ambient temperature measurement corresponding to the first point in time within the time period, and the set of temperature offset values is a first set of temperature offset values also corresponding to the first point in time, wherein the method further comprises:
receiving a second ambient temperature measurement that indicates temperature outside the first cassette at the second point in time within the time period; and
determining a second set of temperature offset values that indicate respective differences between the second ambient temperature measurement and the second set of internal temperature measurements, wherein the second set of temperature offset values correspond to the second point in time,
wherein the first point in time belongs to a first stage of a biological material production process, and the second point in time belongs to a second stage of a biological material production process.

28. The method of any one of claims 21-27, further comprising wirelessly transmitting the set of internal temperature measurements to a computing device.

29. A method of temperature control performed in automated biological material engineering system, the method comprising:
receiving, by a control circuit, a set of internal temperature measurements during a first time period when a first cassette is housed in the automated biological material engineering system, wherein the set of internal temperature measurements indicate temperature within the first cassette, and are generated during the first time period by an array of temperature probes disposed within the first cassette;
receiving, by the control circuit, a first ambient temperature measurement when the first cassette is housed in the automated biological material engineering system, wherein the first ambient temperature measurement indicates temperature outside the first cassette, and is generated during the first time period by a system temperature sensor of the automated biological material engineering system that is disposed outside the first cassette;
determining, by the control circuit, a set of temperature offset values that indicate respective differences between the set of internal temperature measurements and the first ambient temperature measurement;
determining, by the control circuit, a target internal temperature value for a location in a second cassette; and
controlling, by the control circuit during a second time period when the second cassette is housed in the automated biological material engineering system, a heating device or cooling device of the automated biological material engineering system based on the target internal temperature value, the set of temperature offset values, and one or more additional ambient temperature measurement generated by the system temperature sensor during the second time period, wherein the system temperature sensor is disposed outside the second cassette.

30. The method of claim 29, wherein the automated biological material engineering system is an automated cell engineering system, and the target internal temperature value is a desired cell culture temperature value for a cell culture within the second cassette.

31. The method of claim 29 or claim 30, further comprising generating, based on the set of temperature offset values and the one or more additional ambient temperature measurements generated during the second time period, a temperature map that indicates how temperature varies spatially across the second cassette, wherein the heating device or cooling device is controlled based on the temperature map.

32. The method of claim 31, wherein controlling the heating device or cooling device comprises determining an estimated internal temperature value for the location in the second cassette, wherein the heating device or the cooling device is controlled based on a difference between the estimated internal temperature value and the desired internal temperature value.

33. The method of claim 32, wherein controlling the heating device or the cooling device comprises determining a target ambient temperature value based on the estimated internal temperature value and/or the target internal temperature value, wherein the heating device or the cooling device is controlled to cause temperature measured by the system temperature probe to approach the target ambient temperature value.

34. The method of claim 33, wherein the set of temperature offset values is one of multiple sets of temperature offset values determined during the first time period, wherein the multiple sets correspond to different points in time within the first time period,
wherein the determined temperature map corresponds to a point in time within the second time period, and is generated by selecting the set of temperature offset values from among the multiple sets of temperature offset values based on a determination that the set of temperature offset values also corresponds to the point in time within the second time period.

35. The method of claim 34, wherein the set of temperature offset values is associated with a point in time within the first time period, and wherein the point in time within the first time period and the point in time within the second time period both belong to a same stage of a biological material production process that has multiple stages.

36. The method of claim 34 or 35, wherein the temperature map is a first temperature map, the method further comprising:
selecting, at a second point in time within the second time period, a second set of temperature offset values from among the multiple sets of temperature offset values, wherein the selected second set of temperature offset values correspond to the second point in time within the second time period;
generating a second temperature map based on the second set of temperature offset values, wherein the second temperature map is associated with the second point in time within the second time period, and wherein the heating device or the cooling device is controlled based on the second temperature map.

37. The method of claim 36, wherein the target ambient temperature value is a first target ambient temperature value, the method further comprising: determining a second estimated internal temperature value determining a second target ambient temperature value based on the second estimated internal temperature value and/or the target internal temperature value; and
controlling the heating device or the cooling device to cause temperature measured by the system temperature probe to transition from the first target ambient temperature value to the second target ambient temperature value.

38. The method of claim 37, wherein the first target ambient temperature value is higher than the second target ambient temperature value.

39. The method of any one of claims 21-38, wherein the second cassette has no temperature sensor disposed therein.
